(19) United States
(12) Patent Application Publication
(10) Pub. No.: US 2021/0010090 A1
(43) Pub. Date: Jan. 14, 2021

(54) METHOD AND SYSTEM FOR PREDICTING RECURRENCE AND NON-RECURRENCE OF MELANOMA USING SENTINEL LYMPH NODE BIOMARKERS

(71) Applicant: UNIVERSITY OF LOUISVILLE RESEARCH FOUNDATION, INC., Louisville, KY (US)

(72) Inventors: Hongying Hao, Louisville, KY (US); Kelly M. McMasters, Crestwood, KY (US)

(73) Assignee: UNIVERSITY OF LOUISVILLE RESEARCH FOUNDATION, INC., Louisville, KY (US)

(21) Appl. No.: 16/986,324

(22) Filed: Aug. 6, 2020

Related U.S. Application Data
(63) Continuation of application No. 14/401,804, filed on Nov. 17, 2014, now abandoned.
(60) Provisional application No. 61/648,714, filed on May 18, 2012.

(51) Int. Cl. C12Q 1/6886; G01N 33/574

(52) U.S. Cl. CPC C12Q 1/6886; G01N 33/5743; C12Q 2600/112; C12Q 2600/158; G01N 2800/54; C12Q 2600/118

(57) ABSTRACT

A method of characterizing melanoma in a subject involves determining the presence or level of one or more biomarkers in a sample obtained from a sentenal lymph node (SLN) of a subject.

### RELATED APPLICATIONS

This application claims priority from U.S. Provisional Application Ser. No. 61/648,714 filed May 18, 2012, the entire disclosure of which is incorporated herein by this reference.

### TECHNICAL FIELD

The presently disclosed subject matter relates to methods for prognosis of melanoma in a subject. In particular, the presently-disclosed subject matter relates to methods of predicting melanoma recurrence and survival in a subject by determining a presence or level of one or more biomarkers in a sample obtained from a sentinel lymph node of the subject.

### INTRODUCTION

Melanoma is characterized by both a rapidly-rising incidence and a growing lifetime risk. Melanoma treatment is confounded as a rather heterogeneous disease and very wide variability of prognosis, with subsets of patients undergoing unexpectedly poor prognosis. Lack of a precise prognostic tool results in the imprecise application of adjuvant therapy by both under-treatment of those patients who are at high risk of recurrence and over-treatment of those who are actually at low risk.

At present, melanoma prognosis is based on clinicopathologic factors and a population-based staging system. The histological and clinicopathological factors include Breslow thickness, primary tumor ulceration, primary tumor anatomic site (extremities, trunk, head and neck), age, gender, number of positive lymph nodes, the largest diameter of metastatic foci in the sentinel lymph node, and distant metastasis. The standard staging system is the American Joint Committee on Cancer (AJCC) TNM classification. It is based on the combination of 3 factors: (1) tumor thickness (T), as described by Breslow thickness (expressed in millimeters); (2) lymph node status (N); and (3) distant metastasis (M). The TNM staging system identifies 4 stages associated with different clinical outcomes. These histological and clinicopathological prognostic factors should only serve as the primary stratification criteria. There, however, still remains significant variability in overall risk assessment for individual patients.

Sentinel lymph node (SLN) status is the strongest predictor of survival for patients with clinically localized melanoma.¹⁻⁵ Melanoma patients with a single microscopically-positive sentinel lymph node (SLN) are classified as stage III. In the past, when nodal metastases were commonly diagnosed when palpable, the 5-year survival rate for stage III melanoma was approximately 30%. However, SLN biopsy, with intensive histopathological and immunohistochemical analysis, has allowed detection of very early nodal metastasis. Patients classified as stage III and often are advised to undergo expensive and substantially toxic adjuvant therapy. However, the 5-year survival rate of such patients, with or without adjuvant therapy, exceeds 70%, indicating that such patients constitute an “intermediate-risk” rather than “high-risk” group.^{6,7} Clearly, there are limitations in assessing the wide range of prognosis among stage III patients using the current American Joint Committee on Cancer (AJCC) melanoma staging system, which is based solely on clinicopathologic features. Development of precise biomarkers to allow for better assessment of relative risk would have significant clinical utility for individualized treatment.

As such, there is an unmet need for new biomarkers that individually, or in combination with other prognostic factors, allow for prediction of recurrence and survival of melanoma patients to avoid unnecessary treatment in lower risk patients and to improve the clinical outcome in all patients.

### SUMMARY

The presently-disclosed subject matter meets some or all of the above-identified needs, as will become evident to those of ordinary skill in the art after a study of information provided in this document.

The presently-disclosed subject matter includes methods, systems, and kits that make use of biomarkers to allow for better assessment of relative risk of in melanoma subjects.

Most current gene expression research has focused on gene signatures of the primary melanoma itself.⁸⁻¹¹ Uniquely, the presently-disclosed subject matter focuses on biomarkers and biomarker signatures from a sentinel lymph node (SLN). For example, gene expression signatures in node-positive subjects were proposed for diagnostic and prognostic use. Without wishing to be bound by theory or mechanism, exposure of the SLN to melanoma cells is believed to trigger an immune response (or lack thereof) that is reflected in patterns of SLN gene expression (e.g., by examining gene product, including mRNA, protein, etc.). The presently-disclosed subject matter proposes use of a SLN biomarker or biomarker signature for prognosis of melanoma.

In some embodiments, the presently-disclosed subject matter includes a method of prognosticating melanoma in a subject, which includes determining the presence or level of one or more biomarkers in a sample obtained from a sentenal lymph node (SLN) of a subject; and comparing the presence or level of the one or more biomarkers in the sample to a control, wherein a change in the amount of the one or more biomarkers in the sample from the subject, relative to that of the control, is prognostic of the melanoma. In some embodiments the method includes predicting recurrence, nonrecurrence and/or survival in a subject. In some embodiments the method includes identifying a subject as low-risk, intermediate-risk, or high-risk for recurrence.

In some embodiments, the subject has a single positive sentinel lymph node. In some embodiments, the subject is classified as having Stage III melanoma. In some embodiments the method includes identifying a subject classified as having Stage III melanoma as low-risk, intermediate-risk, or high-risk for recurrence.

The presently-disclosed subject matter further includes systems and kits for use in characterizing melanoma in a subject. In some embodiments a kit can include, for example, a probe or a primer for determining the presence or level of each of one or more biomarkers.

In some embodiments of the presently-disclosed subject matter, a method of characterizing melanoma in a subject is provided, and involves determining the presence or level of one or more biomarkers in a sample obtained from a sentenal lymph node (SLN) of a subject; and comparing the presence or level of the one or more biomarkers in the sample to a control, wherein the melanoma is characterized based on a measurable difference in the presence of level of the one or more biomarkers in the sample from the subject as compared to the control.

In some embodiments, the control is selected from: a reference standard, and a level of presence or level of one or more biomarkers in a sample obtained from a sentinel lymph node (SLN) of a control subject.

In some embodiments of the presently-disclosed subject matter, a method for assessing a presence or an amount of one or more biomarkers in a sample obtained from a sentenal lymph node (SLN), involves determining the presence or level of one or more biomarkers in a sample obtained from a SLN of a subject, and comparing the presence or level of the one or more biomarkers in the sample to a control.

In some embodiments, the one or more biomarkers includes TFAP2A. In some embodiments, the one or more biomarkers is selected from ABCB5, TFAP2A, MUC7, PIGR, ERBB3, PAX3, RGS2, and IL1B. In some embodiments, the one or more biomarkers is selected from RGS1, RGS2, PIGR, CD69, ERBB3, and SOD2. In some embodiments, the one or more biomarkers is selected from ABCB5 and MUC7. In some embodiments, the one or more biomarkers is selected from RGS2, PIGR, CD69, SOD2, ABCB5, NR4A2, and MUC7. In some embodiments, the one or more biomarkers is selected from RGS2, PIGR, MUC7, ABCB5, NR4A2.

In some embodiments, the one or more biomarkers is selected from the biomarkers set forth in Tables A, C, D, E, F, and G. In some embodiments, the one or more biomarkers is selected from the biomarkers set forth in Table A. In some embodiments, the one or more biomarkers is selected from the biomarkers set forth in Table C. In some embodiments, the one or more biomarkers is selected from the biomarkers set forth in Table D. In some embodiments, the one or more biomarkers is selected from the biomarkers set forth in Table E. In some embodiments, the one or more biomarkers is selected from the biomarkers set forth in Table F. In some embodiments, the one or more biomarkers is selected from the biomarkers set forth in Table G.

In some embodiments of the method, the comparing step involves comparing the presence or level of the one or more melanoma-associated biomarkers in the sample to a control comprises identifying a biomarker signature of the sample. In some embodiments, the biomarker signature comprises at least 2, 3, 4, 5, 6, 7, 8, 9, 10, 11, 12, 13, 14, 15, 16, 17, 18, 19, 20, 21, 22, 23, 24, or 25 biomarkers. In some embodiments, the biomarker signature comprises at least 2, biomarkers set forth in Table F. In some embodiments, the biomarker signature comprises RGS1, RGS2, PIGR, CD69, ERBB3, and SOD2. In some embodiments, the biomarker signature comprises ABCB5 and MUC7. In some embodiments, the biomarker signature comprises RGS2, PIGR, CD69, SOD2, ABCB5, NR4A2, and MUC7. In some embodiments, the biomarker signature comprises RGS2, PIGR, MUC7, ABCB5, NR4A2.

In some embodiments, the method also includes assessing in the subject a clinicopathologic feature. In some embodiments, the clinicopathologic feature is selected from: age, gender, anatomic location, Breslow thickness, ulceration, and sentinel lymph node status. In some embodiments, the clinicopathologic feature is selected from: metastasis, age, lesion site, tumor burden, number of positive nodes, ulceration, and tumor thickness.

In some embodiments, the method also includes determining the presence or level of one or more biomarkers in a sample obtained from a nonsentinel lymph node of a subject; and comparing the presence or level of the one or more biomarkers in the sample to a control.

In some embodiments of the method, recurrence in the subject is predicted. In some embodiments of the method, nonrecurrence in the subject is predicted. In some embodiments of the method, survival in the subject is predicted. In some embodiments of the method, the subject is identified as low-risk for recurrence. In some embodiments of the method, the subject is identified as intermediate risk for recurrence. In some embodiments of the method, the subject is identified as high risk for recurrence.

In some embodiments of the method, the subject had been, i.e., previously, diagnosed with melanoma. In some embodiments, the subject had been diagnosed with Stage III melanoma.

In some embodiments of the method, determination of the presence or level of one or more biomarkers is conducted using real-time polymerase chain reaction (PCR). In some embodiments, the determination of the presence or level of one or more biomarkers is conducted using a probe for selectively binding each of the one or more biomarkers. In some embodiments, the probe is a nucleotide for hybridizing with the biomarker. In some embodiments, the probe is an antibody for selectively binding the biomarker.

In some embodiments, the method further includes selecting a treatment or modifying a treatment for the melanoma based on the presence or level of the one or more biomarkers. In some embodiments, the method further includes selecting a treatment or modifying a treatment for the melanoma based on the presence or level of the one or more biomarkers and the one or more clinicopatholgic features.

In some embodiments, the method is performed in vitro. In some embodiments, the method is performed ex vivo.

In some embodiments of the presently-disclosed subject matter, a kit is provided, useful for carrying out the methods as described herein. In some embodiments, a kit provided in accordance with the presently-disclosed subject matter includes a probe for determining the presence or level of each of one or more melanoma-associated biomarkers in a sample obtained from a sentinel lymph node (SLN) of a subject. In some embodiments, the probe is selected from a nucleotide and an antibody.

In some embodiments, the kit includes at least 2, 3, 4, 5, 6, 7, 8, 9, 10, 11, 12, 13, 14, 15, 16, 17, 18, 19, 20, 21, 22, 23, 24, 25, 26, 27, 27, 29, or 30 probes for determining the presence or level of each of two or more melanoma-associated biomarkers. In some embodiments, the probe is or the probes are provided on a substrate.

In some embodiments, the it includes a primer pair for determining the presence or level of each of one or more melanoma-associated biomarkers in a sample obtained from a sentinel lymph node (SLN) of a subject. In some embodiments, the kit includes at least 2, 3, 4, 5, 6, 7, 8, 9, 10, 11, 12, 13, 14, 15, 16, 17, 18, 19, 20, 21, 22, 23, 24, 25, 26, 27, 27, 29, or 30 primer pairs for determining the presence or level of each of two or more melanoma-associated biomarkers.

In some embodiments, the kit includes a probe or primer pair for each of one or more biomarkers as set forth in Tables A, C, D, E, F, and G. In some embodiments, the kit includes a probe or primer pair for each of one or more biomarkers as set forth in Table A. In some embodiments, the kit includes probe or primer pair for each of one or more biomarkers as set forth in Table C. In some embodiments, the kit includes a probe or primer pair for each of one or more biomarkers as set forth in Table D. In some embodiments, the kit includes a probe or primer pair for each of one or more biomarkers as set forth in Table E. In some embodiments, the kit includes a probe or primer pair for each of one or more biomarkers as set forth in Table F. In some embodiments, the kit includes a probe or primer pair for each of one or more biomarkers as set forth in Table G.

In some embodiments, the kit includes a probe or primer pair for each of one or more biomarkers selected from ABCB5, TFAP2A, MUC7, PIGR, ERBB3, PAX3, RGS2, and IL1B. In some embodiments, the kit includes a probe or primer pair for each of the following biomarkers: ABCB5, TFAP2A, MUC7, PIGR, ERBB3, PAX3, RGS2, and IL1B. In some embodiments, the kit includes a probe or primer pair for each of one or more biomarkers selected from RGS1, RGS2, PIGR, CD69, ERBB3, and SOD2. In some embodiments, the kit includes a probe or primer pair for each of the following biomarkers: RGS1, RGS2, PIGR, CD69, ERBB3, and SOD2. In some embodiments, the kit includes a probe or primer pair for TFAP2A. In some embodiments, the kit includes a probe or primer pair for each of one or more biomarkers selected from RGS2, PIGR, CD69, SOD2, ABCB5, NR4A2, and MUC7. In some embodiments, the kit includes a probe or primer pair for each of the following biomarkers: RGS2, PIGR, CD69, SOD2, ABCB5, NR4A2, and MUC7. In some embodiments, the kit includes a probe or primer pair for each of one or more biomarkers selected from ABCB5 and MUC7. In some embodiments, the kit includes a probe or primer pair for each of the following biomarkers: ABCB5 and MUC7. In some embodiments, the kit includes a probe or primer pair for each of one or more biomarkers selected from RGS2, PIGR, MUC7, ABCB5, NR4A2. In some embodiments, the kit includes a probe or primer pair for each of the following biomarkers:RGS2, PIGR, MUC7, ABCB5, NR4A2.

In some embodiments, the kit also includes a reference standard sample to obtain a presence or level of the one or more melanoma-associated biomarkers for use as a control to which the sample from the subject can be compared. In some embodiments, the kit also includes control data of a presence or level of the one or more melanoma-associated biomarkers for use as a control to which the sample from the subject can be compared. In some embodiments, the kit also includes reference data for one or more clinicopathologic features.

This Summary describes several embodiments of the presently-disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently-disclosed subject matter, whether listed in this Summary or not. To avoid excessive repetition, this Summary does not list or suggest all possible combinations of such features.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are used, and the accompanying drawings of which:

FIG. 1. Study design. SLN, sentinel lymph node; ROC, receiver operating characteristics; RT-PCR, reverse transcriptase polymerase chain reaction; AUC, area under the receiver operating characteristic curve.

FIG. 2. Heat map diagram and hierarchical-clustering algorithm of 20 differentially expressed genes between the case group and the control group using Euclidean Distance as similarity measure.

FIG. 3. Kaplan-Meier analysis of Disease Free Survival (A, C) and Overall Survival (B, D) according to the 5 SLN gene signature expression level (A, B) and AJCC TNM staging system (C, D), respectively.

### DESCRIPTION OF EXEMPLARY EMBODIMENTS

The presently-disclosed subject matter includes methods, systems, and kits for predicting recurrence and nonrecurrence of melanoma using sentinel lymph node (SLN) biomarkers. The presently-disclosed subject matter further includes methods, systems, and kits for assessing a presence or an amount of one or more biomarkers, e.g., biomarkers associated with melanoma) in a sample obtained from a SLN.

In some embodiments of the presently-disclosed subject matter, a method of prognosticating melanoma in a subject includes determining the presence or level of one or more biomarkers in a sample obtained from a sentinel lymph node (SLN) of a subject; and comparing the presence or level of the one or more biomarkers in the sample to a control, wherein a change in the one or more biomarkers in the sample from the subject, relative to that of the control, is prognostic of the melanoma.

In some embodiments of the presently-disclosed subject matter, a method for assessing a presence or an amount of one or more biomarkers in a sample obtained from a sentenal lymph node (SLN) involves determining the presence or level of one or more biomarkers in a sample obtained from a SLN of a subject, and comparing the presence or level of the one or more biomarkers in the sample to a control. The biomarkers can be associated with melanoma. The sample can be obtained for an SLN of a subject. In some embodiments, the subject had been previously diagnosed with melanoma.

The term “melanoma” is taken to mean a tumor arising from the melanocytic system of the skin and other organs. Melanomas include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma subungal melanoma, and superficial spreading melanoma.

The term “sample” when used to identify a sample obtained from a sentinel lymph node refers to a sample that comprises a biomolecule and/or is derived from a sentinel lymph node of the subject. Representative biomolecules include, but are not limited to total DNA, RNA, miRNA, mRNA, and polypeptides. The biological sample can be used for the detection of the presence and/or expression level of a biomolecule of interest (e.g., biomarker). Any biopsy, tissue, tissue section, cell, group of cells, cell fragment, or cell product from the lymph node can be used with the methods, systems, and kits of the presently claimed subject matter. In some embodiments, the sample can be provided as a frozen or fresh cell or tissue sample (e.g., paraffin-embedded tissue). In some embodiments, the sample can be provided as an extract (e.g., mRNA extracted from cell or tissue).

The terms “comparing” and “correlating,” as used herein in reference to the use of diagnostic and prognostic biomarkers associated with melanoma, refers to comparing the presence or level (quantity) of the biomarker in a subject to its presence or level (quantity) of the biomarker in a control.

The term “control” is used herein to refer to a reference to which a sample can be compared. In some embodiments, the control can be a reference standard. A reference standard can be a manufactured control sample, designed to include a predetermined presence or amount of one or more biomarkers to which a sample can be compared. In some embodiments, a reference standard can comprise a compilation about the presence and/or level of one or more biomarkers considered to be control values. In some embodiments the control can be a sample obtained from a sentinel lymph node of a control subject. A “control subject” can be selected with consideration to the subject being tested. As will be recognized by the skilled artisan, in some embodiments, a control subject can be a subject in which melanoma has not recurred for a period of about 5, 6, 7, 8, 9, 10, or more years. In some embodiments, a control subject can be a subject which has been nonsymptomatic for a period of about 5, 6, 7, 8, 9, 10, or more years. In some embodiments, a control subject can be a subject which is free of melanoma. In some embodiments, the control can be an average or composite value based on analysis of a population of “control subjects.”

In certain embodiments, a biomarker is used to make a particular prognosis by merely its presence (or absence). In other embodiments, a threshold level of a biomarker can be established, and the level of the biomarker in a subject sample can simply be compared to the threshold level. In some embodiments, a biomarker level is used to make a particular prognosis by determining the amount/quantitating the biomarker level.

The term “characterizing” comprises providing a diagnosis, prognosis and/or theranosis. The terms “diagnosing” and “diagnosis” as used herein refer to methods by which the skilled artisan can estimate and even determine whether or not a subject is suffering from a given disease or condition. The skilled artisan often makes a diagnosis on the basis of one or more diagnostic indicators, such as for example a biomarker (e.g., biomarker expression level, biomarker signature), the amount (including presence or absence) of which is indicative of the presence, severity, or absence of the condition.

Along with diagnosis, clinical “prognosis” or “prognosticating” is also an area of great concern and interest. It is important to know the aggressiveness of, relative risk associated with, and the likelihood of recurrence, survival, and nonrecurrence in order to plan the most effective therapy. Some types of melanoma, for example, are managed by several alternative strategies. Current treatment decisions for individual subjects can be based on, for example, (1) tumor thickness, (2) the number of positive lymph nodes involved with disease, (2) cancer marker(s) status, (3) distance of metastasis, and (4) stage of disease at diagnosis. However, even with these factors, accurate prediction of the course of disease for all melanoma subjects is not possible. If a more accurate prognosis can be made, appropriate therapy, and in some instances less severe therapy, for the patient can be chosen. Determination of biomarkers from a sample obtained from a SLN of a subject, as disclosed herein, can be useful in order to categorize subjects according to advancement of melanoma who will benefit from particular therapies and differentiate from other subjects where alternative or additional therapies can be more appropriate. Treatment related diagnostics are sometimes referred to as “theranostics.”

In some embodiments of the presently disclosed subject matter, a method includes categorizing or identifying the subject as low-risk, intermediate-risk, or high risk for recurrence. In some embodiments, recurrence of melanoma in the subject is predicted. In some embodiments recurrence of melanoma within 3, 4, or 5 years is predicted. In some embodiments, survival of the subject is predicted. In some embodiments survival of the subject for 5, 6, 7, 8, 9, 10, 11, 12, 13, 14, or 15 years is predicted. In some embodiments, nonrecurrence of melanoma in the subject is predicted. In some embodiments nonrecurrence of melanoma for 5, 6, 7, 8, 9, 10, 11, 12, 13, 14, or 15 years is predicted.

Making a prognosis or “prognosticating” can refer to predicting a clinical outcome (with or without medical treatment), selecting an appropriate treatment (or whether treatment would be effective), or monitoring a current treatment and potentially changing the treatment, based on the presence or level of one or more biomarkers in a sample obtained from a SLN of the subject. “Prognosticating” as used herein refers to methods by which the skilled artisan can predict the course or outcome of a condition in a subject. The term “prognosis” can refer to the ability to predict the course or outcome of a condition with up to 100% accuracy, or predict that a given course or outcome is more or less likely to occur based on the presence, absence or levels of a biomarker. The term “prognosis” can also refer to an increased probability that a certain course or outcome will occur; that is, that a course or outcome is more likely to occur in a subject exhibiting a given biomarker presence or level, or biomarker signature, when compared to those individuals not exhibiting the given biomarker presence or level, or biomarker signature. For example, in an individual not exhibiting the biomarker expression and/or signature, the chance of a given outcome (e.g., recurrence) may be very low (e.g., <1%), or even absent. In contrast, in individuals exhibiting a different biomarker expression and/or signature, the chance of a given outcome (e.g., recurrence) may be higher. In certain embodiments, a prognosis is about a 5% chance of a given expected outcome, about a 7% chance, about a 10% chance, about a 12% chance, about a 15% chance, about a 20% chance, about a 25% chance, about a 30% chance, about a 40% chance, about a 50% chance, about a 60% chance, about a 75% chance, about a 90% chance, or about a 95% chance.

The skilled artisan will understand that associating a prognostic indicator with a predisposition to an outcome can be performed using statistical analysis. For example, biomarker(s) of greater or less than a control level in some embodiments can signal that a subject is more likely to have a particular outcome (e.g., recurrence) than subjects with a level about equal to the control level, as determined by a level of statistical significance. Statistical significance is often determined by comparing two or more populations, and determining a confidence interval and/or a p value. See, e.g., Dowdy and Wearden, Statistics for Research, John Wiley & Sons, New York, 1983, incorporated herein by reference in its entirety. Exemplary confidence intervals of the present subject matter are 90%, 95%, 97.5%, 98%, 99%, 99.5%, 99.9% and 99.99%, while exemplary p values are 0.1, 0.05, 0.025, 0.02, 0.01, 0.005, 0.001, and 0.0001. When performing multiple statistical tests, e.g., determining differential expression of a panel of biomarker levels, p values can be corrected for multiple comparisons using techniques known in the art.

In other embodiments, a threshold degree of change in the level of a biomarker(s) can be established, e.g., as compared to a control, and the degree of change in the level of the biomarker in a SLN sample can simply be compared to the threshold degree of change in the level. Exemplary threshold change in the level for biomarker(s) of the presently disclosed subject matter can be about 5%, about 10%, about 15%, about 20%, about 25%, about 30%, about 50%, about 60%, about 75%, about 100%, or about 150%. In some embodiments, a “nomogram” can be established, by which a level of a biomarker(s) can be directly related to an associated disposition towards a given outcome. The skilled artisan is acquainted with the use of such nomograms to relate two numeric values with the understanding that the uncertainty in this measurement is the same as the uncertainty in the marker concentration because individual sample measurements are referenced, not population averages.

Biomarkers useful in the context of the presently-disclosed subject matter include those set forth in Tables A, C, D, E, F and G. In some embodiments, the one or more biomarkers can be selected from the biomarkers set forth in Table A, as follows:

TABLE A

ABCB5
PIGR
TFAP2A
MUC7
MLANA
ERBB3
PAX3
DCT
CD69
MOP-1
CD163
IFIT1
DUSP1
THBS1
IL1B
SOD2
PTGS2
RGS2
RGS1
NR4A2

In some embodiments, the one or more biomarkers can be selected from the biomarkers set forth in Table C, set forth herein. In some embodiments, the one or more biomarkers can be selected from the biomarkers set forth in Table D, set forth herein. In some embodiments, the one or more biomarkers can be selected from the biomarkers set forth in Table E, set forth herein. In some embodiments, the one or more biomarkers can be selected from the biomarkers set forth in Table F, set forth herein. In some embodiments, the one or more biomarkers can be selected from the biomarkers set forth in Table G, set forth herein.

In some embodiments, the one or more biomarkers is selected from ABCB5, TFAP2A, IL1B, and PAX3. In some embodiments, the one or more biomarkers includes ABCB5.

As used herein, when a biomarker is identified by a “gene”, “gene symbol” or the like (such as the genes and gene symbols identified in Tables A, C, D, E, F and G), it should be recognized that the biomarker is a product of that gene. A gene product can include, for example, mRNA and protein. As such, biomarkers of the presently-disclosed subject matter include polynucleotides and polypeptides.

The identity and relative quantity of biomarkers in a sample can be used to provide biomarkers profiles or biomarker signatures for a particular sample. A biomarkers signature for a sample can include information about the identities of biomarkers contained in the sample, quantitative levels of biomarkers contained in the sample, and/or changes in quantitative levels of biomarkers relative to another sample or control. For example, a biomarker signature for a sample can include information about the identities, quantitative levels, and/or changes in quantitative levels of biomarkers from a SLN sample from a particular subject. In some embodiments, a biomarker signature relates to information about two or more biomarkers in a sample (e.g., biomarker signature consisting of 2 biomarkers). In some embodiments, a biomarker signature consists of 2, 3, 4, 5, 6, 7, 9, 10, 11, 12, 13, 14, 15, 16, 17, 18, 19, 20, 21, 22, 23, 24, 25, 26, 27, 28, 29, or 30 biomarkers.

In some embodiments, method of the presently-disclosed subject matter further includes assessing in the subject one or more clinicopathologic features. Consideration of clinicopathologic features can in some cases increase specificity and sensitivity of the prognosis. In some embodiments, clinicopathologic features can include, for example, age, gender, anatomic location, Breslow thickness, ulceration, and sentinel lymph node status. In other embodiments, clinicopathologic features can include, for example, metastasis, age, lesion site, tumor burden, number of positive nodes, ulceration, and tumor thickness.

Additional information regarding clinicopathologic features can be found at www.melanomacalculator.com, which information available as of the filing date of this document is incorporated herein by this reference. Also incorporated herein by this reference, and including teachings describing clinicopathologic features and melanoma staging, is Callender G G, Gershenwald J E, Egger M E, Scoggins C R, Martin R C 2nd, Schacherer C W, Edwards M J, Urist M M, Ross M I, Stromberg A J, McMasters K M. “A novel and accurate computer model of melanoma prognosis for patients staged by sentinel lymph node biopsy: comparison with the American Joint Committee on Cancer model.” J Am Coll Surg. 2012 April; 214(4):608-17; discussion 617-9. Epub 2012 Feb. 17. Further information regarding clinicopathologic features can be found in publications by the American Joint Committee on Cancer (AJCC) and about the AJCC staging system. Soong S J, Ding S, Coit D, Balch C M, Gershenwald J E, Thompson J F, Gimotty P; AJCC Melanoma Task Force. “Predicting survival outcome of localized melanoma: an electronic prediction tool based on the AJCC Melanoma Database.” Ann Surg Oncol. 2010 August; 17(8):2006-14. Epub 2010 Apr. 9. In this regard, information can be found at www.melanomaprognosis.org, which information available as of the filing date of this document is incorporated herein by this reference. Also incorporated herein by this reference are D. Collett. Modelling Survival Data in Medical Research. London: Chapman & Hall, 1994; and Ding S, Soong S J, Lin H Y, Desmond R, Balch C M. Parametric modeling of localized melanoma prognosis and outcome. J Biopharm Statistics, 19(4):732-47, 2009.

As noted above with reference to clinicopathologic features, it is contemplated that other information can be useful for increasing sensitivity and specificity of embodiments of the methods described herein. For example, in some embodiments, a method can further include determining the presence or level of one or more biomarkers in a sample obtained from a nonsentinel lymph node of a subject; and comparing the presence or level of the one or more biomarkers in the sample to a control.

Further with respect to the diagnostic and prognostic methods of the presently disclosed subject matter, a preferred subject is a vertebrate subject. A preferred vertebrate is warm-blooded; a preferred warm-blooded vertebrate is a mammal. A mammal is most preferably a human. As used herein, the term “subject” includes both human and animal subjects. Thus, veterinary therapeutic uses are provided in accordance with the presently disclosed subject matter. As such, the presently disclosed subject matter provides for the diagnosis and prognosis of mammals such as humans, as well as those mammals of importance due to being endangered, such as Siberian tigers; of economic importance, such as animals raised by humans; and/or animals of social importance to humans, such as animals kept as pets or in zoos. Examples of such animals include but are not limited to: carnivores such as cats and dogs; swine, including pigs, hogs, and wild boars; ruminants and/or ungulates such as cattle, oxen, sheep, giraffes, deer, goats, bison, and camels; and horses (including race horses). In some embodiments, the subject is a human.

In some embodiments, the subject has a single positive sentinel lymph node. In some embodiments, the subject is classified or diagnosed with stage III melanoma. Classification with stage III melanoma can occur when there is a presence of at least one positive sentinel lymph node. Additional information regarding staging can be found at www.melanomacalculator.com, which information available as of the filing date of this document is incorporated herein by this reference. Also incorporated herein by this reference, and including teachings describing clinicopathologic features and melanoma staging, is Callender G G, Gershenwald J E, Egger M E, Scoggins C R, Martin R C 2nd, Schacherer C W, Edwards M J, Urist M M, Ross M I, Stromberg A J, McMasters K M. “A novel and accurate computer model of melanoma prognosis for patients staged by sentinel lymph node biopsy: comparison with the American Joint Committee on Cancer model.” J Am Coll Surg. 2012 April; 214(4):608-17; discussion 617-9. Epub 2012 Feb. 17. Further information can be found in publications by the American Joint Committee on Cancer (AJCC) and about the AJCC staging system. In this regard, information can be found at www.melanomaprognosis.org, which information available as of the filing date of this document is incoroporated herein by this reference. Also incorporated herein by this reference are D. Collett. Modelling Survival Data in Medical Research. London: Chapman & Hall, 1994; and Ding S, Soong S J, Lin H Y, Desmond R, Balch C M. Parametric modeling of localized melanoma prognosis and outcome. J Biopharm Statistics, 19(4):732-47, 2009.

It is contemplated that the sample obtained from the SLN, or the SLN from which the sample is obtained would be typically acquired at a time when sentinel nodes would be normally identified and removed, for example at or around the time of surgery to remove a primary melanoma. In some cases, it can be desirable to use a fresh sample, or a paraffin-embedded tissue sample. In some cases, it can be desirable to freeze or otherwise store for use at a later date. In some cases, it can be useful to process (e.g., extract) the sample, using a portion for immediate testing and/or saving a portion for use at a later date.

As noted hereinabove, the presently disclosed subject matter provides for the determination of the presence or level of a biomarker(s) in a SLN sample. The presence or level of one or more biomarkers of interest in the sample can then be determined using any of a number of methodologies generally known in the art and compared to biomarker control levels.

An exemplary methodology for measuring biomarker levels from a SLN is microarray technique. The technique provides many polynucleotides with known sequence information as probes to find and hybridize with the complementary strands in a sample to thereby capture the complementary strands by selective binding. In some cases, a microarray can provide many probes for selectively binding proteins (e.g., antibodies).

The term “selective binding” as used herein refers to a measure of the capacity of a probe to hybridize to a target polynucleotide or to bind a target polypeptide with specificity. Thus, in the case of a target polynucleotide, the probe comprises a polynucleotide sequence that is complementary, or essentially complementary, to at least a portion of the target polynucleotide sequence. Nucleic acid sequences which are “complementary” are those which are base-pairing according to the standard Watson-Crick complementarity rules. As used herein, the term “complementary sequences” means nucleic acid sequences which are substantially complementary, as can be assessed by the same nucleotide comparison set forth above, or as defined as being capable of hybridizing to the nucleic acid segment in question under relatively stringent conditions such as those described herein. A particular example of a contemplated complementary nucleic acid segment is an antisense oligonucleotide. With regard to probes disclosed herein having binding affinity to mRNAs, the probe can be 100% complementary with the target polynucleotide sequence. However, the probe need not necessarily be completely complementary to the target polynucleotide along the entire length of the target polynucleotide so long as the probe can bind the target polynucleotide with specificity and capture it from the sample.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by the skilled artisan. Stringent temperature conditions will generally include temperatures in excess of 30° C., typically in excess of 37° C., and preferably in excess of 45° C. Stringent salt conditions will ordinarily be less than 1,000 mM, typically less than 500 mM, and preferably less than 200 mM. However, the combination of parameters is much more important than the measure of any single parameter. Determining appropriate hybridization conditions to identify and/or isolate sequences containing high levels of homology is well known in the art. For the purposes of specifying conditions of high stringency, preferred conditions are a salt concentration of about 200 mM and a temperature of about 45° C.

Data mining work is completed by bioinformatics, including scanning chips, signal acquisition, image processing, normalization, statistic treatment and data comparison as well as pathway analysis. As such, microarray can profile hundreds and thousands of polynucleotides simultaneously with high throughput performance. Microarray profiling analysis of mRNA expression has successfully provided valuable data for gene expression studies in basic research. And the technique has been further put into practice in the pharmaceutical industry and in clinical diagnosis.

The analysis of biomarkers correlated with melanoma can be carried out separately or simultaneously with multiple probes within one test sample (e.g., multiple polynucleotide or polypeptide probes). For example, several probes can be combined into one test for efficient processing of a multiple of samples and for potentially providing greater prognostic accuracy. In addition, one skilled in the art would recognize the value of testing multiple samples from the same subject.

In some embodiments, a panel consisting of biomarker probes that selectively bind biomarkers, as described herein, to provide relevant information related to the prognosis of a subject. Such a panel can be constructed, for example, using 1, 2, 3, 4, 5, 6, 7, 8, 9, 10, 15, 20, 30, 40, 50, 75, 100, 150, 200, 250, 300, 400, 500, or 1,000 individual biomarker probes. The analysis of a single probe or subsets of probes comprising a larger panel of probes could be carried out by one skilled in the art to optimize clinical sensitivity or specificity in various clinical settings. These include, but are not limited to ambulatory, urgent care, critical care, intensive care, monitoring unit, in-subject, out-subject, physician office, medical clinic, and health screening settings. Furthermore, one skilled in the art can use a single probe or a subset of additional probes comprising a larger panel of probes in combination with an adjustment of the diagnostic threshold in each of the aforementioned settings to optimize clinical sensitivity and specificity. The clinical sensitivity of an assay is defined as the percentage of those with non-recurrence, recurrence and/or survival that the assay correctly predicts.

In some embodiments, determining the amount of the one or more biomarkers comprises labeling the one or more biomarkers. The labeled biomarkers can then be captured with one or more probes that each selectively binds the one or more biomarkers.

As used herein, the terms “label” and “labeled” refer to the attachment of a moiety, capable of detection by spectroscopic, radiologic, or other methods, to a probe molecule. Thus, the terms “label” or “labeled” refer to incorporation or attachment, optionally covalently or non-covalently, of a detectable marker into/onto a molecule, such as a polynucleotide or polypeptide. Various methods of labeling polynucleotides and polypeptides are known in the art and can be used. Examples of labels for biomarkers include, but are not limited to, the following: radioisotopes, fluorescent labels, heavy atoms, enzymatic labels or reporter genes, chemiluminescent groups, biotinyl groups, predetermined polypeptide epitopes recognized by a secondary reporter (e.g., leucine zipper pair sequences, binding sites for antibodies, metal binding domains, epitope tags, etc.). In some embodiments, labels are attached by spacer arms of various lengths to reduce potential steric hindrance.

The analysis of biomarkers levels using probes can be carried out in a variety of physical formats as well. For example, the use of microtiter plates or automation can be used to facilitate the processing of large numbers of test samples. Alternatively, single sample formats could be developed to facilitate immediate treatment and diagnosis in a timely fashion.

In some embodiments, the plurality of probes are each bound to a substrate. In some embodiments, the substrate comprises a plurality of addresses. Each address can be associated with at least one of the probes of the array. An array is “addressable” when it has multiple regions of different moieties (e.g., different polynucleotide sequences) such that a region (i.e., a “feature” or “spot” of the array) at a particular predetermined location (i.e., an “address”) on the array will detect a particular target or class of targets (although a feature may incidentally detect non-targets of that feature). Array features are typically, but need not be, separated by intervening spaces. In the case of an array, the “target” biomarker can be referenced as a moiety in a mobile phase (typically fluid), to be detected by probes (“target probes”) which are bound to the substrate at the various regions.

Biopolymer arrays (e.g., polynucleotide microarrays) can be fabricated by depositing previously obtained biopolymers (such as from synthesis or natural sources) onto a substrate, or by in situ synthesis methods. Methods of depositing obtained biopolymers include, but are not limited to, loading then touching a pin or capillary to a surface, such as described in U.S. Pat. No. 5,807,522 or deposition by firing from a pulse jet such as an inkjet head, such as described in PCT publications WO 95/25116 and WO 98/41531, and elsewhere. The in situ fabrication methods include those described in U.S. Pat. No. 5,449,754 for synthesizing peptide arrays, and in U.S. Pat. No. 6,180,351 and WO 98/41531 and the references cited therein for polynucleotides, and may also use pulse jets for depositing reagents. Further details of fabricating biopolymer arrays by depositing either previously obtained biopolymers or by the in situ method are disclosed in U.S. Pat. Nos. 6,242,266, 6,232,072, 6,180,351, and 6,171,797. In fabricating arrays by depositing previously obtained biopolymers or by in situ methods, typically each region on the substrate surface on which an array will be or has been formed (“array regions”) is completely exposed to one or more reagents. For example, in either method the array regions will often be exposed to one or more reagents to form a suitable layer on the surface that binds to both the substrate and biopolymer or biomonomer. In in situ fabrication the array regions will also typically be exposed to the oxidizing, deblocking, and optional capping reagents. Similarly, particularly in fabrication by depositing previously obtained biopolymers, it can be desirable to expose the array regions to a suitable blocking reagent to block locations on the surface at which there are no features from non-specifically binding to target.

Determining the amount of biomarkers from SLNs of subjects can alternatively, or in addition to microarray analysis, comprise using real-time polymerase chain reaction (PCR). Real-time PCR (RT-PCR) can provide accurate and rapid data as to presence and amount of mRNAs present in a sample. When conducting RT-PCR analysis, an initial step is the isolation of mRNA from the sample. The starting material is typically total RNA isolated from a SLN. mRNA can be extracted, for example, from frozen or archived paraffin-embedded and fixed (e.g. formalin-fixed) tissue samples.

General methods for mRNA extraction are well known in the art and are disclosed in standard textbooks of molecular biology. RNA isolation can be performed using purification kit, buffer set and protease from commercial manufacturers according to the manufacturer's instructions. For example, total RNA from cells in culture can be isolated using Qiagen RNeasy mini-columns. Numerous RNA isolation kits are commercially available and can be used in the methods of the invention.

One of the first steps in gene expression profiling by RT-PCR is the reverse transcription of the RNA template into cDNA, followed by amplification in a PCR reaction. Commonly used reverse transcriptases include, but are not limited to, avilo myeloblastosis virus reverse transcriptase (AMV-RT) and Moloney murine leukemia virus reverse transcriptase (MMLV-RT). The reverse transcription step is typically primed using specific primers, random hexamers, or oligo-dT primers, depending on the circumstances and the goal of expression profiling. For example, extracted RNA can be reverse-transcribed using a GeneAmp RNA PCR kit (Perkin Elmer, Calif., USA), following the manufacturer's instructions. The derived cDNA can then be used as a template in the subsequent PCR reaction.

Although the PCR step can use a variety of thermostable DNA-dependent DNA polymerases, it typically employs the Taq DNA polymerase, which has a 5′-3′ nuclease activity but lacks a 3′-5′ proofreading endonuclease activity. TaqMan PCR typically utilizes the 5′-nuclease activity of Taq or Tth polymerase to hydrolyze a hybridization probe bound to its target amplicon, but any enzyme with equivalent 5′ nuclease activity can be used. Two oligonucleotide primers are used to generate an amplicon typical of a PCR reaction. A third oligonucleotide, or probe, is designed to detect nucleotide sequence located between the two PCR primers. The probe is non-extendible by Taq DNA polymerase enzyme, and is labeled with a reporter fluorescent dye and a quencher fluorescent dye. Any laser-induced emission from the reporter dye is quenched by the quenching dye when the two dyes are located close together as they are on the probe. During the amplification reaction, the Taq DNA polymerase enzyme cleaves the probe in a template-dependent manner The resultant probe fragments disassociate in solution, and signal from the released reporter dye is free from the quenching effect of the second fluorophore. One molecule of reporter dye is liberated for each new molecule synthesized, and detection of the unquenched reporter dye provides the basis for quantitative interpretation of the data.

TaqMan™ RT-PCR can be performed using commercially available equipment, such as, for example, ABI PRISM 7700™ Sequence Detection System™ (Perkin-Elmer-Applied Biosystems, Foster City, Calif., USA), or Lightcycler (Roche Molecular Biochemicals, Mannheim, Germany). In one specific embodiment, the 5′ nuclease procedure is run on a real-time quantitative PCR device such as the ABI PRISM 7700™ Sequence Detection System™. The system consists of a thermocycler, laser, charge-coupled device (CCD), camera and computer. The system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

5′-Nuclease assay data are initially expressed as Ct, or the threshold cycle. As discussed above, fluorescence values are recorded during every cycle and represent the amount of product amplified to that point in the amplification reaction. The point when the fluorescent signal is first recorded as statistically significant is the threshold cycle (Ct).

To minimize errors and the effect of sample-to-sample variation, RT-PCR is usually performed using an internal standard. The ideal internal standard is expressed at a constant level among different tissues, and is unaffected by the experimental treatment. RNAs most frequently used to normalize patterns of gene expression are mRNAs for the housekeeping genes glyceraldehyde-3-phosphate-dehydrogenase (GAPDH), Beta-2-microglobulin (B2M), and β-actin.

Another variation of the RT-PCR technique is the real time quantitative PCR, which measures PCR product accumulation through a dual-labeled fluorigenic probe (i.e., TaqMan™ probe). Real time PCR is compatible both with quantitative competitive PCR, where internal competitor for each target sequence is used for normalization, and with quantitative comparative PCR using a normalization gene contained within the sample, or a housekeeping gene for RT-PCR.

The practice of the presently disclosed subject matter can employ, unless otherwise indicated, conventional techniques of cell biology, cell culture, molecular biology, transgenic biology, microbiology, recombinant DNA, and immunology, which are within the skill of the art. Such techniques are explained fully in the literature. See e.g., Molecular Cloning A Laboratory Manual (1989), 2nd Ed., ed. by Sambrook, Fritsch and Maniatis, eds., Cold Spring Harbor Laboratory Press, Chapters 16 and 17; U.S. Pat. No. 4,683,195; DNA Cloning, Volumes I and II, Glover, ed., 1985; Oligonucleotide Synthesis, M. J. Gait, ed., 1984; Nucleic Acid Hybridization, D. Hames & S. J. Higgins, eds., 1984; Transcription and Translation, B. D. Hames & S. J. Higgins, eds., 1984; Culture Of Animal Cells, R. I. Freshney, Alan R. Liss, Inc., 1987; Immobilized Cells And Enzymes, IRL Press, 1986; Perbal (1984), A Practical Guide To Molecular Cloning; See Methods In Enzymology (Academic Press, Inc., N.Y.); Gene Transfer Vectors For Mammalian Cells, J. H. Miller and M. P. Calos, eds., Cold Spring Harbor Laboratory, 1987; Methods In Enzymology, Vols. 154 and 155, Wu et al., eds., Academic Press Inc., N.Y.; Immunochemical Methods In Cell And Molecular Biology (Mayer and Walker, eds., Academic Press, London, 1987; Handbook Of Experimental Immunology, Volumes I-IV, D. M. Weir and C. C. Blackwell, eds., 1986.

In some embodiments of the presently-disclosed subject matter, methods can additionally include selecting a treatment or modifying a treatment for the melanoma based on the presence or level of the one or more biomarkers. For example, if a subject is determined as low-risk, intermediate-risk, or high risk, an appropriate treatment can be selected of a treatment can be appropriately modified based on the risk stratification.

The presently-disclosed subject matter further includes systems and kits, which are useful for practicing embodiments of the methods as described herein. In some embodiments a kit is provided, which includes a reagent to carry out the method of any preceding claim.

In some embodiments, the presently-disclosed subject matter includes a system or kit for use in characterizing melanoma in a subject, which includes a probe for determining the presence or level of each of one or more melanoma-associated biomarkers in a sample obtained from a sentinel lymph node (SLN) of a subject. In some embodiment, the probe(s) are polynucleotides. In some embodiments, the probe(s) are antibodies. In some embodiments, the probe(s) is provided on a substrate. In some embodiments the kit includes a probe for each of at least 2, 3, 4, 5, 6, 7, 8, 9, 10, 11, 12, 13, 14, 15, 16, 17, 18, 19, 20, 21, 22, 23, 24, 25, 26, 27, 27, 29, or 30 melanoma-associated biomarkers.

In some embodiments, the presently-disclosed subject matter includes a system or kit for use in characterizing melanoma in a subject, which includes a primer pair for determining the presence or level of each of one or more melanoma-associated biomarkers in a sample obtained from a sentinel lymph node (SLN) of a subject. In some embodiments, the system includes at least 2, 3, 4, 5, 6, 7, 8, 9, 10, 11, 12, 13, 14, 15, 16, 17, 18, 19, 20, 21, 22, 23, 24, 25, 26, 27, 27, 29, or 30 primer pairs for determining the presence or level of each of two or more melanoma-associated biomarkers.

In some embodiments, systems and kits of the presently-disclosed subject matter further include a reference standard sample to obtain a presence or level of the one or more melanoma-associated biomarkers for use as a control to which the sample from the subject can be compared. In some embodiments, the systems and kits further include control data of a presence or level of the one or more melanoma-associated biomarkers for use as a control to which the sample from the subject can be compared. In some embodiments, the systems and kits further include reference data for one or more clinicopathologic features.

The details of one or more embodiments of the presently-disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

While the terms used herein are believed to be well understood by one of ordinary skill in the art, definitions are set forth herein to facilitate explanation of the presently-disclosed subject matter.

Unless defined otherwise, all technical and scientific terms used herein have the same meaning as commonly understood by one of ordinary skill in the art to which the presently-disclosed subject matter belongs. Although any methods, devices, and materials similar or equivalent to those described herein can be used in the practice or testing of the presently-disclosed subject matter, representative methods, devices, and materials are now described.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, properties such as reaction conditions, and so forth used in the specification and claims are to be understood as being modified in all instances by the term “about”. Accordingly, unless indicated to the contrary, the numerical parameters set forth in this specification and claims are approximations that can vary depending upon the desired properties sought to be obtained by the presently-disclosed subject matter.

As used herein, the term “about,” when referring to a value or to an amount of mass, weight, time, volume, concentration or percentage is meant to encompass variations of in some embodiments ±20%, in some embodiments ±10%, in some embodiments ±5%, in some embodiments ±1%, in some embodiments ±0.5%, and in some embodiments ±0.1% from the specified amount, as such variations are appropriate to perform the disclosed method.

As used herein, ranges can be expressed as from “about” one particular value, and/or to “about” another particular value. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

The presently-disclosed subject matter is further illustrated by the following specific but non-limiting examples. The following examples may include compilations of data that are representative of data gathered at various times during the course of development and experimentation related to the present invention. The following examples include prophetic examples.

### EXAMPLES

### Example 1

Patients and Methods

Patient information. De-identified human subject data from the Sunbelt Melanoma Trial database were used in this study. The trial was a randomized, prospective trial enrolled over 3600 patients between 1997 and 2003, involving 79 centers throughout North America. It was approved by the institutional review board (IRB) at each institution. Eligibility criteria included patients aged 18 to 70 years, invasive melanomas ≥1.0 mm Breslow thickness, and no clinical evidence of regional or distant metastasis. All patients were staged with SLN biopsy. All SLN samples were obtained after subjects had provided written informed consent. Patients underwent excision of the primary melanoma and SLN biopsy using intradermal injection of technetium sulfur colloid around the primary tumor site. A lymphoscintigram was obtained and a hand-held gamma probe was used intraoperatively to guide SLN identification. Intradermal injection of isosulfan blue dye (1 to 5 mL) was performed in the majority of patients as well. All blue nodes and all nodes ≥10% of the most radioactive or hottest node were collected as SLNs.⁴² A histologically positive SLN was defined as evidence of metastatic tumor cells identified by either hematoxylin and eosin (H and E) or immunohistochemistry (IHC). A central pathology review committee re-evaluated all cases of tumor-positive SLN samples to assure that they are true-positive SLNs. This study only included patients with positive lymph nodes in the Sunbelt Melanoma Trial. These node-positive patients were randomized to receive adjuvant interferon-alfa-2b (IFN) therapy versus no adjuvant therapy. IFN had no statistically significant differences in disease-free survival (DFS) or overall survival (OS) for node-positive patients and SLN was obtained before treatment.⁴⁵. Therefore, the entire cohort can be considered a homogeneous group. Data regarding clinicopathologic factors, recurrence, and survival were collected prospectively. Additional details of the Sunbelt Melanoma Trial have been described elsewhere.⁴³

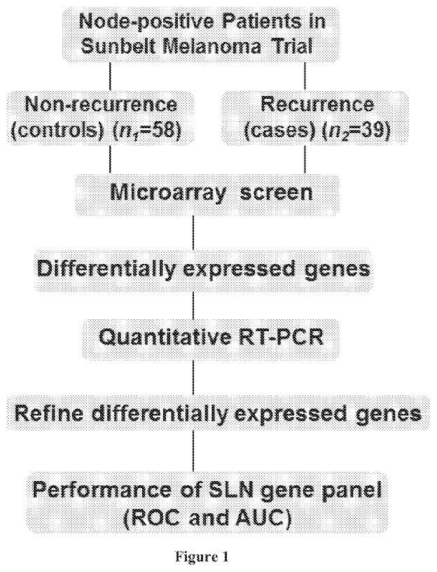

Study design. Reference is made to FIG. 1. All of the SLN-positive samples used in this study were from the Sunbelt Melanoma Trial. The present inventors defined the control group as those who had no recurrence after 5 years (with 58 samples, n₁=58). The case group included who experienced recurrence within 5 years (with 39 samples, n₂=39). The median (range) follow-up is 79 months (6.0 months-122.0 months). Table B shows demographic and clinicopathologic data for all 97 node-positive subjects selected from the Sunbelt Melanoma Trial.

The primary end point was disease-free survival (DFS), defined as the time from the date of random assignment to the date of the first recurrence. Overall survival (OS) was calculated from the date of random assignment to the date of death.

TABLE B
Clinicopathological characteristics in node-positive
patients from the Sunbelt Melanoma Trial (n = 97)

| | Group | | | | | | |
|---|---|---|---|---|---|---|---|
| | | | No Recurrence | | | | |
| | Total | | and Alive | | Recurrence | | |
| Variables | (N = 97) | | (N = 58) | | (N = 39) | | P Value |
| Gender | | | | | | | 0.774 |
| Female (%) | 44 | (45.4) | 27 | (46.6) | 17 | (43.6) | |
| Male (%) | 53 | (54.6) | 31 | (53.4) | 22 | (56.4) | |
| Age (years) | | | | | | | 0.069 |
| 19-44 (%) | 43 | (44.3) | 30 | (51.7) | 13 | (33.3) | |
| 45-54 (%) | 22 | (22.7) | 14 | (24.1) | 8 | (20.5) | |
| 55-71 (%) | 32 | (33.0) | 14 | (24.1) | 18 | (46.2) | |
| Age (years) | | | | | | | 0.014 |
| Mean (95% CI) | 46.9 | (44.3-49.5) | 44.3 | (41.1-47.5) | 50.9 | (46.7-55.1) | |
| Median (min − max) | 46.0 | (19.0-71.0) | 43.5 | (19.0-69.0) | 54.0 | (25.0-71.0) | |
| Primary tumor site | | | | | | | 0.553 ^{†} |
| Head (%) | 3 | (3.1) | 2 | (3.4) | 1 | (2.6) | |
| Lower Extremity (%) | 28 | (28.9) | 14 | (24.1) | 14 | (35.9) | |
| Neck (%) | 2 | (2.1) | 1 | (1.7) | 1 | (2.6) | |
| Trunk (%) | 47 | (48.5) | 32 | (55.2) | 15 | (38.5) | |
| Upper Extremity (%) | 16 | (16.5) | 8 | (13.8) | 8 | (20.5) | |
| Histological Subtype | | | | | | | 0.104 ^{†} |
| Acral lentinginous | 8 | (8.2) | 2 | (3.4) | 6 | (15.4) | |
| melanoma (%) | | | | | | | |
| Lentigo maligna | 2 | (2.1) | 1 | (1.7) | 1 | (2.6) | |
| melanoma (%) | | | | | | | |
| Nodular melanoma (%) | 32 | (33.0) | 17 | (29.3) | 15 | (38.5) | |
| Other (%) | 14 | (14.4) | 9 | (15.5) | 5 | (12.8) | |
| Superficial spreading | 37 | (38.1) | 27 | (46.6) | 10 | (25.6) | |
| melanoma (%) | | | | | | | |
| Breslow Thickness (cm) | | | | | | | 0.024 |
| Mean (95% CI) | 2.9 | (2.5-3.3) | 2.6 | (2.2-2.9) | 3.5 | (2.7-4.3) | |
| Median (min − max) | 2.2 | (1.0-13.0) | 2.0 | (1.0-6.8) | 2.5 | (1.1-13.0) | |
| Clark Level | | | | | | | |
| II/III (%) | 14 | (14.4) | 9 | (15.8) | 5 | (12.8) | 0.686 |
| IV/V (%) | 82 | (84.5) | 48 | (84.2) | 34 | (87.2) | |
| Ulceration Present | | | | | | | |
| NA (%) | 2 | (2.1) | 1 | (1.7) | 1 | (2.6) | 0.191 |
| No (%) | 57 | (58.8) | 37 | (63.8) | 20 | (51.3) | |
| Yes (%) | 37 | (38.1) | 19 | (32.8) | 18 | (46.2) | |
| Positive SLN Count | | | | | | | |
| 1 (%) | 79 | (81.4) | 46 | (79.3) | 33 | (84.6) | 0.510 |
| >1 (%) | 18 | (18.6) | 12 | (20.7) | 6 | (15.4) | |
| SLN Count | | | | | | | |
| 1 (%) | 16 | (16.5) | 10 | (17.2) | 6 | (15.4) | 0.809 |
| >1 (%) | 81 | (83.5) | 48 | (82.8) | 33 | (84.6) | |
| Total Positive LN | | | | | | | |
| 1 (%) | 74 | (76.3) | 43 | (74.1) | 31 | (79.5) | 0.544 |
| >1 (%) | 23 | (23.7) | 15 | (25.9) | 8 | (20.5) | |
| Follow-up Time (All | | | | | | | <.001 |
| Patients) | | | | | | | |
| (months) | | | | | | | |
| Mean (95% CI) | 77.4 | (71.8-83.0) | 87.0 | (81.5-92.5) | 63.2 | (53.5-72.9) | |
| Median (min − max) | 79.0 | (6.0-122.0) | 93.0 | (40.0-122.0) | 58.0 | (6.0-122.0) | |
| Follow-up Time (Censored | | | | | | | 0.071 |
| Patients) (months) | | | | | | | |
| Mean (95% CI) | 89.0 | (84.0-94.1) | 87.0 | (81.5-92.5) | 99.7 | (88.5-111.0) | |
| Median (min − max) | 94.0 | (40.0-122.0) | 93.0 | (40.0-122.0) | 99.0 | (73.0-122.0) | |

Microarray and qRT-PCR. GeneChip Human HG-U133 plus 2.0 array (Affymetrix, Santa Clara, CA) was used to screen candidate differential expressed genes in recurrence versus non-recurrence group.

For qRT-PCR experiments, total SLN RNA (100 ng) from each sample was reverse transcribed with the SuperScript III First-Strand Synthesis System. mRNA primers were purchased from Life Technologies (Carlsbad, Calif.). Quantitative RT-PCR reactions were completed on a 7500 Fast Real Time PCR system (Life Technologies). The relative quantity of the target mRNA was normalized to endogenous gene (GAPDH or B2M). The fold changes were calculated with the 2^{-ΔΔCt} method.

RNA isolation and microarray analysis. A portion of each SLN (defined as one fourth of the lymph node or a 2-mm³ portion of the node, whichever was smaller) was snap-frozen on dry ice or liquid nitrogen and stored at −80° C. until it was shipped on dry ice. If more than one SLN was found, each SLN was processed identically. The remaining SLN tissue was processed by hematoxylin and eosin (H and E) staining at multiple levels, with at least five sections per block, along with two additional random sections for S-100 immunohistochemistry (IHC). Most of these node-positive patients have small micrometastases (<2 mm); the majority of the SLN tissue sampled contains lymph node cells, not melanoma cells. SLNs were processed and analyzed by a central laboratory (National Genetics Institute) that was blinded to clinical and pathologic data. Total RNA was extracted using TRIREAGENT (Molecular Research Center Inc, Cincinnati, Ohio).⁴⁴ Traces of DNA contamination was removed by DNase digestion before applying to the array. RNA quality control/quantity assessment (QA/QC) was checked by Agilent bioanalyzer and Nanodrop ND-1000 respectively prior to microarray experiments. RNA samples with a minimum RNA integrity number (RIN) of at least 7 were selected. Total RNAs from each sample were labeled, fragmented, and then hybridized with Affymetrix GeneChip Human HG-U133 plus 2.0 array according to the manufacturer's guidelines. The quality control of the microarray experiments is checked by the percent present of 40% to 60% and 3′/5′ ratio of control probe sets (Actin and GAPDH) less than 3.

Statistical analysis. For microarray analysis, a fold change outlier filter was applied to reduce the dimension of the data before determining differentially expressed genes (DEGs) between the controls and the cases.^{18, 19} That is, using fold change (FC) to determine which genes should be tested for differential expression. For each of 54,675 probes on the array, the FC was calculated and four filters, T1={μ(FC)±1.5σ(FC)}, T2={μ(FC)±2σ(FC)}, T3={μ(FC)±3σ(FC)} and T4={μ(FC)±4σ(FC)}, were used to filter data, where μ(FC) is the mean of fold changes and σ(FC) is the standard deviation of fold changes from all 54,675 probes. The genes that fell inside T1, T2 and T3 were filtered from the differential data respectively. After filtering the data, t-test for normal gene expression data and Wilcoxon test for non-normal expression data were applied²⁰. In order to control for multiplicity, the Benjamini-Hochberg method¹⁹ was employed to adjust the p-values. The present inventors chose to use the filter T3=μ(FC)±3σ(FC) for analysis. To predict the group of recurrence or non-recurrence, the multiple logistic regression model is employed. For the data obtained by qRT-PCR, the fold changes were calculated with the 2^{-ΔΔCt} method of recurrence group versus non-recurrence group. Data are presented as mean±SD. A p value of <0.05 was considered to be statistically significant. The predicted probability of recurrence was used as a surrogate marker to construct receiver operating characteristic (ROC) curve. Area under the ROC curve (AUC) was used as an accuracy index for evaluating the prognostic performance of the selected SLN gene panel. All p values were two sided. Survival distributions were estimated using Kaplan-Meier methods and the log-rank test was used to assess the statistical significance of differences in DFS and OS between groups.

Results

Patient characteristics. Reference is made to Table B. For all patients, there was a significant difference in the mean follow-up time between the non-recurrence group (87 months) and the recurrence group (63.2 months) (p<0.001). For censored patients, there was no significant difference in the mean follow-up time between the non-recurrence group (87 months) and the recurrence group (99.7 months) (p=0.071). There were no significant differences between the case group and the control group in the distribution of gender; age groups of 19-44, 45-54, and 55-71; primary tumor site; histological subtype; Clark level; presence of ulceration; number of positive SLN; total number of SLN removed; SLN count; or total positive lymph node count. Therefore, the case and control groups were equally balanced for further SLN gene signature analysis regarding all of these factors. Factors that were significantly associated with recurrence were increasing age and Breslow thickness (p=0.014. and 0.024, respectively).

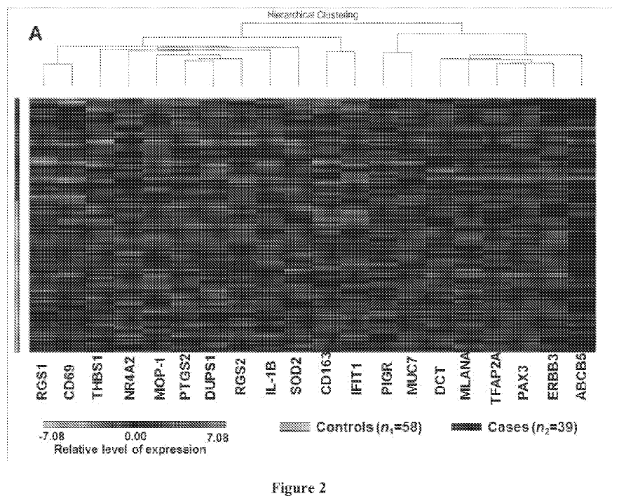

Differentially expressed SLN gene profiles in the case group versus control group. The Affymetrix GeneChip Human HG-U133 Plus 2.0 array has more than 47,000 transcripts and variants, including approximately 38,500 well-characterized human genes. This array was used to screen the differentially expressed SLN genes between the case group and the control group in this cohort (n=97). Using filter T3, the present inventors detected 213 differentially expressed probe sets with a p<0.05. The present inventors used Partek Genomics Suite v6.5 to annotate those probe sets. There were 52 probe sets without defined gene names. The present inventors further removed those probe sets with lower fold changes (−1.5<fold change<2). There were 8 genes with significantly higher expression levels in the case group than those in the control group (fold change>2.0, p<0.05). There were 12 genes with significantly lower expression levels in the case group than in the control group (fold change<−1.5, p<0.05). These 20 differentially expressed genes were identified as candidates for further testing via RT-PCR (Table C). A heat map diagram and hierarchical-clustering algorithm illustrated the differentially expressed SLN genes in the case group and the control group according to the genes' patterns of expression (FIG. 2). These results showed that distinctive SLN gene profiles are present in node-positive melanoma patients.

TABLE C
Twenty differentially expressed SLN genes in the case group versus the control group.

| | Adjusted | Fold | Gene | |
|---|---|---|---|---|
| Probe ID | P value | Change | Symbol | Gene Name |
| 240717_at | 0.035 | 13.2826 | ABCB5 | ATP-binding cassette, sub-family B |
| | | | | (MDR/TAP), member 5 |
| 226147_s_at | 0.0206 | 9.2597 | PIGR | Polymeric immunoglobulin receptor |
| 217059_at | 0.0498 | 6.4195 | MUC7 | Mucin 7, secreted |
| 210669_at | 0.0352 | 3.079 | TFAP2A | Transcription factor AP-2 alpha (activating |
| | | | | enhancer binding protein 2 alpha) |
| 205337_at | 0.0392 | 2.9196 | DCT | Dopachrome tautomerase (dopachrome |
| | | | | delta-isomerase, tyrosine-related protein 2) |
| 206426_at | 0.0495 | 2.4934 | MLANA | Melan-A |
| 202454_s_at | 0.0248 | 2.0885 | ERBB3 | v-erb-b2 erythroblastic leukemia viral |
| | | | | oncogene homolog 3 (avian) |
| 231666_at | 0.0495 | 2.0681 | PAX3 | Paired box 3 |
| 209795_at | 0.0217 | −1.5034 | CD69 | CD69 molecule |
| 224277_at | 0.0328 | −1.5384 | MOP-1 | MOP-1 |
| 215049_x_at | 0.0416 | −1.5734 | CD163 | CD163 molecule |
| 203153_at | 0.0392 | −1.6034 | IFIT1 | Interferon-induced protein with |
| | | | | tetratricopeptide repeats 1 |
| 201041_s_at | 0.0448 | −1.702 | DUSP1 | Dual specificity phosphatase 1 |
| 201110_s_at | 0.0392 | −1.7262 | THBS1 | Thrombospondin 1 |
| 205067_at | 0.0333 | −1.7426 | IL1B | Interleukin 1, beta |
| 215078_at | 0.0257 | −1.7454 | SOD2 | Superoxide dismutase 2, mitochondrial |
| 204748_at | 0.0392 | −1.7709 | PTGS2 | Prostaglandin-endoperoxide synthase 2 |
| | | | | (prostaglandin G/H synthase and |
| | | | | cyclooxygenase) |
| 202388_at | 0.0196 | −1.8651 | RGS2 | Regulator of G-protein signaling 2, 24 kDa |
| 216834_at | 0.0095 | −1.94 | RGS1 | Regulator of G-protein signaling 1 |
| 204621_s_at | 0.0475 | −2.0379 | NR4A2 | Nuclear receptor subfamily 4, group A, |
| | | | | member 2 |

Confirming and refining the SLN gene panel RT-PCR was performed to confirm the 20 SLN genes with significantly different expression levels between the case and the control groups identified by microarray analysis. Most of these differentially expressed genes were confirmed by RT-PCR (Table D). This result validated the microarray result. This SLN gene panel was used for the prediction of melanoma recurrence or non-recurrence.

TABLE D
Twenty differentially expressed SLN genes (case group
vs the control group) confirmed by real time RT-PCR.

| Gene Symbol | Microarray Fold Change | PCR Fold Change |
|---|---|---|
| ABCB5 | 13.2826 | 11.6 ± 4.6 |
| PIGR | 9.2597 | 4.5 ± 1.45 |
| MUC7 | 6.4195 | 6.36 ± 1.43 |
| TFAP2A | 3.079 | 4.28 ± 0.67 |
| DCT | 2.9196 | 2.52 ± 1.06 |
| MLANA | 2.4934 | 3.78 ± 0.45 |
| ERBB3 | 2.0885 | 7.78 ± 0.87 |
| PAX3 | 2.0681 | 5.39 ± 0.21 |
| CD69 | −1.5034 | 2.045 ± 0.87 |
| MOP-1 | −1.5384 | −1.96 ± 0.3 |
| CD163 | −1.5734 | −1.62 ± 0.29 |
| IFIT1 | −1.6034 | −2.1 ± 0.46 |
| DUSP1 | −1.702 | −1.53 ± 0.39 |
| THBS1 | −1.7262 | −1.8 ± 0.07 |
| IL1B | −1.7426 | −1.9 ± 0.61 |
| SOD2 | −1.7454 | −1.65 ± 0.39 |
| PTGS2 | −1.7709 | −1.67 ± 0.37 |
| RGS2 | −1.8651 | −1.59 ± 0.39 |
| RGS1 | −1.94 | −1.78 ± 0.3 |
| NR4A2 | −2.0379 | −1.9 ± 0.4 |

Performance of the individually differentially expressed SLN genes by AUC. In order to compare the weight of risk assessment value of each individual gene, the prognostic accuracy was calculated by AUC. Table E listed the individual AUC for each of the 20 differentially expressed genes. The individual AUCs of the SLN genes range from 0.6379 (DUSP1) to 0.706 (RGS1).

TABLE E
Area Under the Receiver Operating Characteristic Curve (AUCs)
of the twenty differentially expressed SLN genes.

| Gene Symbol | AUC |
|---|---|
| RGS1 | 0.706 |
| RGS2 | 0.7025 |
| PIGR | 0.6958 |
| CD69 | 0.6897 |
| ERBB3 | 0.6839 |
| SOD2 | 0.6813 |
| MOP-1 | 0.668 |
| IL1B | 0.668 |
| ABCB5 | 0.6645 |
| TFAP2A | 0.6627 |
| DCT | 0.6578 |
| IFIT1 | 0.6583 |
| PTGS2 | 0.6583 |
| NR4A2 | 0.6446 |
| PAX3 | 0.6428 |
| MLANA | 0.6424 |
| MUC7 | 0.6419 |
| CD163 | 0.6415 |
| THBS1 | 0.6401 |
| DUSP1 | 0.6379 |

Establishing the prognostic SLN gene panel. Because malignant melanoma is heterogenous, in terms of its biological, immunological, and metastatic properties, and melanoma cells exhibit a polymorphous expression of tumor markers, the power of multiplexed biomarker assays is believed to be greater than that obtainable with any single marker. Furthermore, the multi-marker assay could potentially prevent a high proportion of errors emanating from a single marker. The present inventors sought to identify a robust prognostic SLN gene panel as an independent indicator. Nine genes were selected from the 20 differentially expressed SLN genes. Among them, six genes have an AUC of more than 0.68, which are RGS1, RGS2, PIGR, CD69, ERBB3, and SOD2. Two genes have the highest fold changes, which are ABCB5 and MUC7. One gene had the lowest fold change, which is NR4A2. The prognostic performance for the different combinations of the 9 SLN genes was evaluated. Combination AUCs of more than 0.85 are listed in Table F. The highest AUC obtained was 0.8537 with a combination of 7 genes (RGS2, PIGR, CD69, SOD2, ABCB5, NR4A2, and MUC7). Considering the future clinical application, the present inventors chose a combination of 5 SLN gene panel (RGS2, PIGR, MUC7, ABCB5, NR4A2) with an AUC of 0.8526. The present inventors consider these 5 SLN genes as a core prognostic SLN gene panel.

TABLE F
Combinations of the 9 SLN genes with AUCs of more than 0.85.

| genel | gene2 | gene3 | gene4 | gene5 | gene6 | gene7 | gene8 | gene9 | AUC |
|---|---|---|---|---|---|---|---|---|---|
| G2 | G3 | G4 | G6 | G7 | G9 | G8 | | | 0.8537 |
| G2 | G3 | G4 | G5 | G6 | G7 | G9 | G8 | | 0.8535 |
| G2 | G3 | G4 | G7 | G9 | G8 | | | | 0.8535 |
| G2 | G3 | G4 | G5 | G7 | G9 | G8 | | | 0.8532 |
| G2 | G3 | G5 | G6 | G7 | G9 | G8 | | | 0.853 |
| G2 | G3 | G5 | G7 | G9 | G8 | | | | 0.8526 |
| G2 | G3 | G7 | G9 | G8 | | | | | 0.8526 |
| G1 | G2 | G3 | G4 | G5 | G6 | G9 | G8 | | 0.8521 |
| G2 | G3 | G6 | G7 | G9 | G8 | | | | 0.8521 |
| G1 | G2 | G3 | G4 | G5 | G6 | G7 | G9 | G8 | 0.8519 |
| G1 | G2 | G3 | G4 | G6 | G7 | G9 | G8 | | 0.8519 |
| G1 | G2 | G3 | G4 | G6 | G9 | G8 | | | 0.8517 |
| G2 | G3 | G4 | G5 | G6 | G9 | G8 | | | 0.8517 |
| G2 | G3 | G4 | G6 | G9 | G8 | | | | 0.8512 |
| G2 | G3 | G5 | G6 | G9 | G8 | | | | 0.8512 |
| G1 | G2 | G3 | G4 | G5 | G7 | G9 | G8 | | 0.851 |
| G1 | G2 | G3 | G4 | G5 | G9 | G8 | | | 0.851 |
| G1 | G2 | G3 | G4 | G7 | G9 | G8 | | | 0.851 |
| G1 | G2 | G3 | G5 | G7 | G9 | G8 | | | 0.8506 |
| G1 | G2 | G3 | G7 | G9 | G8 | | | | 0.8506 |
| G2 | G3 | G4 | G5 | G9 | G8 | | | | 0.8504 |
| G1 | G2 | G3 | G4 | G9 | G8 | | | | 0.8501 |
| G2 | G3 | G5 | G9 | G8 | | | | | 0.8501 |

Gene Names

| | |
|---|---|
| G1 = G216834_at | RGS1 |
| G2 = G202388_at | RGS2 |
| G3 = G226147_s_at | PIGR |
| G4 = G209795_at | CD69 |
| G5 = G202454_s_at | ERBB3 |
| G6 = G215078_at | SOD2 |
| G7 = G240717_at | ABCB5 |
| G8 = G217059_at | MUC7 |
| G9 = G204621_s_at | NR4A2 |

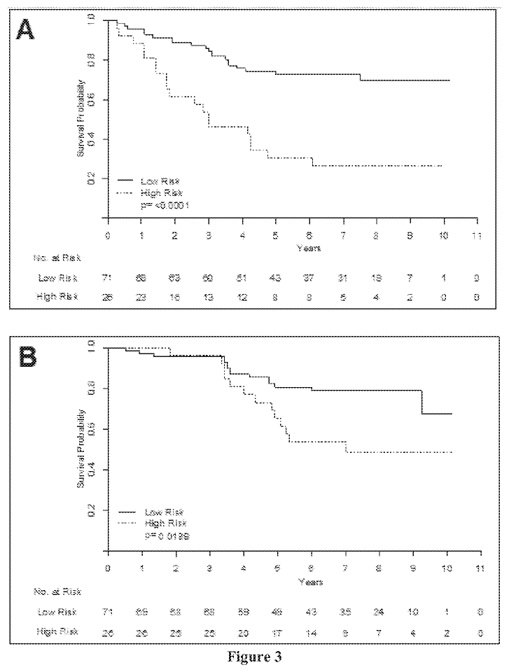

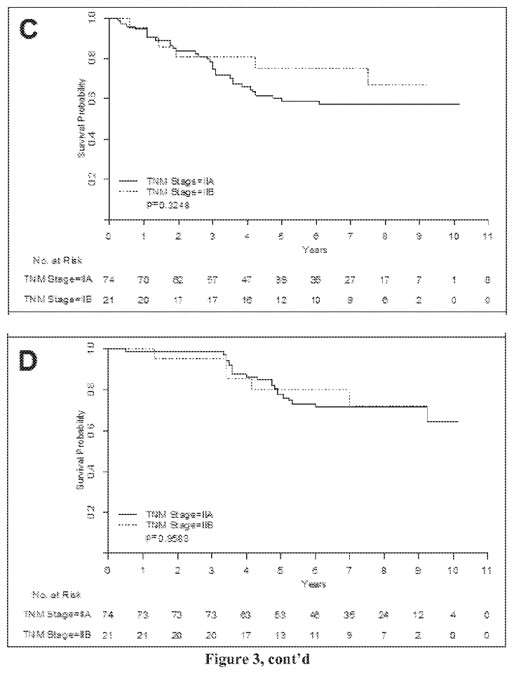

Independent prognostic value of the SLN gene signature. After the present inventors identified the 5 core SLN gene signatures, the present inventors were interested in exploring whether this gene profile can differentiate the truly high-risk from the low-risk melanoma patients. Among the 5 SLN core genes, PIGR, MUC7, and ABCB5 were upregulated, while RGS2 and NR4A2 were downregulated. For each patient, if each of the upregulated gene expression data was more than the average of that upregulated gene, the present inventors gave a score of 1 for that individual upregulated gene. Otherwise the score was 0. If each of the downregulated gene expression data was less than the average of that downregulated gene, the present inventors gave a score of 1 for that individual downregulated gene. Otherwise the score was 0. Each patient's risk score is the sum of the 5 individual gene scores. If the score was 0˜2, then the present inventors defined this patient as a low-risk patient. If the score was 3˜5, then the present inventors defined this patient as a high-risk patient. Using the 5 SLN core gene signature, there were significant differences in DFS (p<0.0001) and OS (p=0.0139) between high-risk and low-risk patients (FIGS. 3A and 3B).

The present inventors next compared the performance of the risk assessment of the 5 SLN core gene signature with that of the traditional AJCC TNM staging system. Among the 97 subjects, 79 patients had 1 positive SLN (stage IIIA), 17 patients had 2 to 3 positive SLNs (stage IIIB), and 1 patient had more than 3 positive SLNs (stage IIIC). The present inventors performed survival analysis on the stage IIIA and IIIB patients, omitting the IIIC patients because of small sample size. The result showed that there were no significant differences in DFS (p=0.3248) and OS (p=0.9583) between stage IIIA and stage IIIB patients based on TNM staging system (FIGS. 3C and 3D). These results showed that the 5 SLN gene signature performed better than that of the AJCC TNM staging system, in terms of differentiation of the high- and low-risk node-positive melanoma patients.

TABLE G
Differentially expressed SLN genes in node-positive melanoma patients.

| | | | Gene | |
|---|---|---|---|---|
| ID | AdjP | FC | Symbol | Gene Title |
| 240717_at | 0.035 | 13.2826 | ABCB5 | ATP-binding cassette, sub-family B |
| | | | | (MDR/TAP), member 5 |
| 222689_at | 0.0495 | −1.2669 | ACER3 | alkaline ceramidase 3 |
| 209612_s_at | 0.0392 | 1.0725 | ADH1B | alcohol dehydrogenase 1B (class I), beta |
| | | | | polypeptide |
| 209160_at | 0.0429 | −1.2498 | AKR1C3 | aldo-keto reductase family 1, member C3 |
| | | | | (3-alpha hydroxysteroid dehydrogenase, |
| | | | | type II) |
| 228573_at | 0.035 | −1.2856 | ANTXR2 | anthrax toxin receptor 2 |
| 221234_s_at | 0.0406 | −1.3092 | BACH2 | BTB and CNC homology 1, basic leucine |
| | | | | zipper transcription factor 2 |
| 1552841_s_at | 0.0095 | 1.2174 | BCL2L14 | BCL2-like 14 (apoptosis facilitator) |
| 1564499_at | 0.0423 | 1.1841 | C14orf81 | chromosome 14 open reading frame 81 |
| 156075l_at | 0.028 | 1.2225 | C18orf16 | chromosome 18 open reading frame 16 |
| 1552895_a_at | 0.0423 | 1.3838 | C21orf99 | cancer-testis SP-1 |
| 1569332_at | 0.0466 | 1.2793 | C3orf66 | chromosome 3 open reading frame 66 |
| 1553886_at | 0.028 | 1.1746 | CCDC108 | coiled-coil domain containing 108 |
| 209924_at | 0.0495 | −1.4002 | CCL18 | chemokine (C-C motif) ligand 18 |
| | | | | (pulmonary and activation-regulated) |
| 205114_s_at | 0.0374 | −1.478 | CCL3/// | chemokine (C-C motif) ligand 3///chemokine |
| | | | CCL3L1/// | (C-C motif) ligand 3-like 1///chemokine |
| | | | CCL3L3 | |
| 1405_i_at | 0.0416 | −1.3007 | CCL5 | chemokine (C-C motif) ligand 5 |
| 215049_x_at | 0.0416 | −1.5734 | CD163 | CD163 molecule |
| 206749_at | 0.0423 | −1.4457 | CD1B | CD1b molecule |
| 215784_at | 0.0423 | −1.3306 | CD1E | CD1e molecule |
| 227458_at | 0.0482 | −1.2616 | CD274 | CD274 molecule |
| 206680_at | 0.0328 | −1.4747 | CD5L | CD5 molecule-like |
| 209795_at | 0.0217 | −1.5034 | CD69 | CD69 molecule |
| 205758_at | 0.0461 | −1.3782 | CD8A | CD8a molecule |
| 205288_at | 0.0391 | −1.3201 | CDC14A | CDC14 cell division cycle 14 homolog A |
| | | | | (S. cerevisiae) |
| 206210_s_at | 0.0481 | −1.3073 | CETP | cholesteryl ester transfer protein, plasma |
| 214596_at | 0.028 | 1.9986 | CHRM3 | cholinergic receptor, muscarinic 3 |
| 223737_x_at | 0.0258 | 1.2246 | CHST9 | carbohydrate (N-acetylgalactosamine 4-0) |
| | | | | sulfotransferase 9 |
| 214598_at | 0.0206 | 1.2845 | CLDN8 | claudin 8 |
| 1552552_s_at | 0.0427 | −1.269 | CLEC4C | C-type lectin domain family 4, member C |
| 1555756_a_at | 0.035 | −1.4215 | CLEC7A | C-type lectin domain family 7, member A |
| 206914_at | 0.028 | −1.3647 | CRTAM | cytotoxic and regulatory T cell molecule |
| 202901_x_at | 0.0171 | −1.485 | CTSS | cathepsin S |
| 204470_at | 0.0392 | −1.3846 | CXCL1 | chemokine (C-X-C motif) ligand 1 (melanoma |
| | | | | growth stimulating activity, alpha) |
| 209774_x_at | 0.0423 | −1.3681 | CXCL2 | chemokine (C-X-C motif) ligand 2 |
| 205337_at | 0.0392 | 2.9196 | DCT | dopachrome tautomerase (dopachrome delta- |
| | | | | isomerase, tyrosine-related protein 2) |
| 242319_at | 0.0254 | 1.2566 | DGKG | diacylglycerol kinase, gamma 90 kDa |
| 225502_at | 0.0466 | −1.256 | DOCK8 | dedicator of cytokinesis 8 |
| 213068_at | 0.047 | −1.2951 | DPT | dermatopontin |
| 204646_at | 0.0392 | −1.3536 | DPYD | dihydropyrimidine dehydrogenase |
| 219597_s_at | 0.0206 | 1.5982 | DUOX1 | dual oxidase 1 |
| 201041_s_at | 0.0448 | −1.702 | DUSP1 | dual specificity phosphatase 1 |
| 209457_at | 0.0461 | −1.3833 | DUSP5 | dual specificity phosphatase 5 |
| 228116_at | 0.0188 | 1.4118 | DUXAP10 | Double homeobox A pseudogene 10 |
| | | | | v-erb-b2 erythroblastic leukemia viral |
| 202454_s_at | 0.0248 | 2.0885 | ERBB3 | oncogene homolog 3 (avian) |
| 215257_at | 0.035 | 1.2403 | ERGIC3 | ERGIC and golgi 3 |
| 230276_at | 0.0206 | −1.3266 | FAM49A | family with sequence similarity 49, member A |
| 1559513_a_at | 0.0392 | 1.3782 | FANCC | Fanconi anemia, complementation group C |
| 227265_at | 0.028 | −1.3329 | FGL2 | fibrinogen-like 2 |
| | | | | ARP3 actin-related protein 3 homolog B |
| 224424_x_at | 0.0495 | 1.4143 | FKSG73 | pseudogene |
| 241953_at | 0.0206 | 1.445 | FLJ25694/// | hypothetical protein FLJ25694///keratin |
| | | | KRTAP21-1/// | associated protein 21-1///hypothetical LOC7 |
| | | | LOC731932 | |
| 217487_x_at | 0.03 | 1.765 | FOLH1 | folate hydrolase (prostate-specific membrane |
| | | | | antigen) 1 |
| 1557122_s_at | 0.0257 | 1.2398 | GABRB2 | gamma-aminobutyric acid (GABA) A |
| | | | | receptor, beta 2 |
| 230766_at | 0.0442 | −1.2937 | GART | phosphoribosylglycinamide formyltransferase, |
| | | | | phosphoribosylglycinamide synthetase, phos |
| 202270_at | 0.0188 | −1.3929 | GBP1 | guanylate binding protein 1, interferon- |
| | | | | inducible, 67 kDa |
| 219777_at | 0.028 | −1.3093 | GIMAP6 | GTPase, IMAP family member 6 |
| 1560142_at | 0.0416 | 1.282 | GRIK2 | glutamate receptor, ionotropic, kainate 2 |
| 203817_at | 0.0374 | −1.3021 | GUCY1B3 | guanylate cyclase 1, soluble, beta 3 |
| 235458_at | 0.0095 | −1.3635 | HAVCR2 | hepatitis A virus cellular receptor 2 |
| 203260_at | 0.0448 | −1.1842 | HDDC2 | HD domain containing 2 |
| 210331_at | 0.0257 | 1.2031 | HECW1 | HECT, C2 and WW domain containing E3 |
| | | | | ubiquitin protein ligase 1 |
| 219403_s_at | 0.0206 | −1.3125 | HPSE | heparanase |
| 210029_at | 0.0095 | −1.402 | IDO1 | indoleamine 2,3-dioxygenase 1 |
| 203153_at | 0.0392 | −1.6034 | IFIT1 | interferon-induced protein with |
| | | | | tetratricopeptide repeats 1 |
| 61732_r_at | 0.0416 | −1.3159 | IFT74 | intraflagellar transport 74 homolog |
| | | | | (Chlamydomonas) |
| 205992_s_at | 0.035 | −1.2956 | IL15 | interleukin 15 |
| 207072_at | 0.0217 | −1.2816 | IL18RAP | interleukin 18 receptor accessory protein |
| 205067_at | 0.0333 | −1.7426 | IL1B | interleukin 1, beta |
| 209821_at | 0.0356 | −1.3672 | IL33 | interleukin 33 |
| 206502_s_at | 0.0427 | −1.3275 | INSM1 | insulinoma-associated 1 |
| 222827_s_at | 0.0423 | 1.329 | KBTBD10 | kelch repeat and BTB (POZ) domain |
| | | | | containing 10 |
| 206765_at | 0.028 | −1.3498 | KCNJ2 | potassium inwardly-rectifying channel, |
| | | | | subfamily J, member 2 |
| 203991_s_at | 0.0452 | −1.3254 | KDM6A | lysine (K)-specific demethylase 6A |
| 1559022_at | 0.0392 | −1.2854 | KIAA0494 | KIAA0494 |
| 1558293_at | 0.0232 | −1.2706 | KIAA1107 | KIAA1107 |
| 223756_at | 0.0188 | 1.2728 | KIAA1310 | KIAA1310 |
| 206785_s_at | 0.0254 | −1.3798 | KLRC1/// | killer cell lectin-like receptor subfamily C, |
| | | | KLRC2 | member 1///killer cell lectin-like rece |
| 207723_s_at | 0.028 | −1.3981 | KLRC3 | killer cell lectin-like receptor subfamily C, |
| | | | | member 3 |
| 207795_s_at | 0.0095 | −1.4395 | KLRD1 | killer cell lectin-like receptor subfamily D, |
| | | | | member 1 |
| 205821_at | 0.0206 | −1.3675 | KLRK1 | killer cell lectin-like receptor subfamily K, |
| | | | | member 1 |
| 34764_at | 0.028 | −1.2893 | LARS2 | leucyl-tRNA synthetase 2, mitochondrial |
| 212446_s_at | 0.0196 | −1.327 | LASS6 | LAG1 homolog, ceramide synthase 6 |
| 230865_at | 0.0423 | −1.3494 | LIX1 | Lix1 homolog (chicken) |
| 216359_at | 0.0392 | 9.6982 | LOC100129410 | hypothetical LOC100129410 |
| 239612_at | 0.0423 | 1.3576 | LOC100240734 | hypothetical LOC100240734 |
| 1558202_at | 0.0206 | 1.2158 | LOC145783 | hypothetical LOC145783 |
| 231828_at | 0.0352 | −1.4116 | LOC253039 | hypothetical LOC253039 |
| 1561225_at | 0.028 | 1.2497 | LOC338579 | hypothetical protein LOC338579 |
| 227940_at | 0.0329 | −1.1919 | LOC339803 | Hypothetical protein LOC339803 |
| 1556992_at | 0.0248 | 1.2219 | LOC550113 | hypothetical LOC550113 |
| 226748_at | 0.0232 | −1.2351 | LYSMD2 | LysM, putative peptidoglycan-binding, |
| | | | | domain containing 2 |
| 204058_at | 0.0427 | −1.2975 | ME1 | malic enzyme 1, NADP(+)-dependent, |
| | | | | cytosolic |
| 239468_at | 0.0206 | 1.2337 | MKX | mohawk homeobox |
| 206426_at | 0.0495 | 2.4934 | MLANA | melan-A |
| 224277_at | 0.0328 | −1.5384 | MOP-1 | MOP-1 |
| 217059_at | 0.0498 | 6.4195 | MUC7 | mucin 7, secreted |
| 225847_at | 0.0308 | −1.3777 | NCEH1 | neutral cholesterol ester hydrolase 1 |
| 203413_at | 0.0427 | −1.3296 | NELL2 | NEL-like 2 (chicken) |
| 231798_at | 0.0475 | −1.3078 | NOG | noggin |
| 204621_s_at | 0.0475 | −2.0379 | NR4A2 | nuclear receptor subfamily 4, group A, |
| | | | | member 2 |
| 204105_s_at | 0.0217 | −1.3562 | NRCAM | neuronal cell adhesion molecule |
| 1555377_at | 0.0321 | 1.1792 | OR4D2 | olfactory receptor, family 4, subfamily D, |
| | | | | member 2 |
| 206637_at | 0.0188 | −1.4158 | P2RY14 | purinergic receptor P2Y, G-protein coupled, |
| | | | | 14 |
| 233705_at | 0.0172 | 1.3326 | PACSIN2 | protein kinase C & casein kinase substrate in |
| | | | | neurons 2 |
| 231666_at | 0.0495 | 2.0681 | PAX3 | paired box 3 |
| 203708_at | 0.0282 | −1.3295 | PDE4B | phosphodiesterase 4B, cAMP-specific |
| | | | | (phosphodiesterase E4 dunce homolog, |
| | | | | Drosophila) |
| 226147_s_at | 0.0206 | 9.2597 | PIGR | polymeric immunoglobulin receptor |
| 215129_at | 0.0333 | 1.5669 | PIK3C2G | phosphoinositide-3-kinase, class 2, gamma |
| | | | | polypeptide |
| 244315_at | 0.0206 | −1.3515 | PLSCR1 | phospholipid scramblase 1 |
| 202166_s_at | 0.0392 | −1.2547 | PPP1R2 | protein phosphatase 1, regulatory (inhibitor) |
| | | | | subunit 2 |
| 228656_at | 0.0416 | −1.4643 | PROX1 | prospero homeobox 1 |
| 219938_s_at | 0.0427 | −1.2504 | PSTPIP2 | proline-serine-threonine phosphatase |
| | | | | interacting protein 2 |
| 1555520_at | 0.0317 | 1.2708 | PTCH1 | patched homolog 1 (Drosophila) |
| 204897_at | 0.0416 | −1.3178 | PTGER4 | prostaglandin E receptor 4 (subtype EP4) |
| 204748_at | 0.0392 | −1.7709 | PTGS2 | prostaglandin-endoperoxide synthase 2 |
| | | | | (prostaglandin G/H synthase and |
| | | | | cyclooxygenase) |
| 1554999_at | 0.0206 | −1.3719 | RASGEF1B | RasGEF domain family, member 1B |
| 232549_at | 0.0392 | −1.2091 | RBM11 | RNA binding motif protein 11 |
| 216834_at | 0.0095 | −1.94 | RGS1 | regulator of G-protein signaling 1 |
| 223809_at | 0.0333 | −1.3615 | RGS18 | regulator of G-protein signaling 18 |
| 202388_at | 0.0196 | −1.8651 | RGS2 | regulator of G-protein signaling 2, 24 kDa |
| 223168_at | 0.0461 | −1.4454 | RHOU | ras homolog gene family, member U |
| 222663_at | 0.0248 | −1.3368 | RIOK2 | RIO kinase 2 (yeast) |
| 210432_s_at | 0.0258 | −1.4924 | SCN3A | sodium channel, voltage-gated, type III, alpha |
| | | | | subunit |
| 222717_at | 0.0427 | −1.306 | SDPR | serum deprivation response |
| 202833_s_at | 0.0188 | −1.4025 | SERPINA1 | serpin peptidase inhibitor, clade A (alpha-1 |
| | | | | antiproteinase, antitrypsin), member 1 |
| 1554027_a_at | 0.0095 | 1.2051 | SLC4A4 | solute carrier family 4, sodium bicarbonate |
| | | | | cotransporter, member 4 |
| 203373_at | 0.0448 | −1.2963 | SOCS2 | suppressor of cytokine signaling 2 |
| 215078_at | 0.0257 | −1.7454 | SOD2 | superoxide dismutase 2, mitochondrial |
| 1569638_at | 0.0403 | 1.4179 | SOX5 | SRY (sex determining region Y)-box 5 |
| 1553851_at | 0.0392 | −1.231 | SPIC | Spi-C transcription factor (Spi-1/PU. 1 related) |
| 1554676_at | 0.0452 | −1.4653 | SRGN | serglycin |
| 216639_at | 0.0185 | 1.186 | SRPX2 | sushi-repeat-containing protein, X-linked 2 |
| 242943_at | 0.0495 | −1.2843 | ST8SIA4 | ST8 alpha-N-acetyl-neuraminide alpha-2,8- |
| | | | | sialyltransferase 4 |
| 221393_at | 0.0392 | 1.2541 | TAAR3 | trace amine associated receptor 3 |
| | | | | (gene/pseudogene) |
| 242388_x_at | 0.0328 | −1.2968 | TAGAP | T-cell activation RhoGTPase activating |
| | | | | protein |
| 210669_at | 0.0352 | 3.079 | TFAP2A | transcription factor AP-2 alpha (activating |
| | | | | enhancer binding protein 2 alpha) |
| 209278_s_at | 0.0328 | −1.3583 | TFPI2 | tissue factor pathway inhibitor 2 |
| 201110_s_at | 0.0392 | −1.7262 | THBS1 | thrombospondin 1 |
| 1552280_at | 0.0392 | −1.4898 | TIMD4 | T-cell immunoglobulin and mucin domain |
| | | | | containing 4 |
| 237132_at | 0.0329 | 1.2031 | TJP2 | tight junction protein 2 (zona occludens 2) |
| 221060_s_at | 0.0352 | −1.328 | TLR4 | toll-like receptor 4 |
| 224496_s_at | 0.028 | −1.4185 | TMEM107 | transmembrane protein 107 |
| 214329_x_at | 0.0391 | −1.3528 | TNFSF10 | tumor necrosis factor (ligand) superfamily, |
| | | | | member 10 |
| 217143_s_at | 0.0206 | −1.3387 | TRA@/// | T cell receptor alpha locus///T cell receptor |
| | | | TRD@ | delta locus |
| 238520_at | 0.0352 | −1.2362 | TRERF1 | transcriptional regulating factor 1 |
| 236972_at | 0.048 | 1.7481 | TRIM63 | tripartite motif-containing 63 |
| 218807_at | 0.0254 | −1.2724 | VAV3 | vav 3 guanine nucleotide exchange factor |
| 217649_at | 0.0392 | −1.2629 | ZFAND5 | zinc finger, AN1-type domain 5 |
| 1557322_at | 0.035 | −1.2765 | ZNF230 | zinc finger protein 230 |
| 214751_at | 0.0188 | −1.2896 | ZNF468 | zinc finger protein 468 |

### Example 2

A prognostic model incorporating a novel SLN gene signature in combination with well-established clinicopathologic factors is further developed and refined. It is anticipated that the prognostic scoring system described herein will overcome the existing barrier in the accurate determination of stage III melanoma prognosis. Importantly, such a prognostic model would also provide much-needed risk stratification to identify homogeneous risk groups for entry of patients into clinical trials of novel adjuvant therapies.

A prognostic scoring system is contemplated, incorporating gene expression signatures in the SLN along with well-characterized clinicopathologic prognostic factors that will predict prognosis among patients with tumor-positive SLN.

By identifying a “low-risk” group of patients who may avoid toxic and expensive adjuvant therapy, patient quality of life will be improved. By identifying a truly “high-risk” group of patients, adjuvant therapy can be focused on the patients most likely to benefit from it.

While there are many biomarkers that have been proposed to predict prognosis in melanoma, the presently-disclosed subject matter is distinct because, none of those studies has been used to stratify risk in patients with nodal metastasis. The overall impact of this work is expected to ensure the best quality of life of melanoma patients by allowing personalized assessment of risk that can drive adjuvant therapy decisions.

Rationale for using SLN as a biomarker: The SLN reflects the status of the entire regional nodal basin and is the most likely site of early metastasis.^{3-5,12,13} SLN status is the single most important prognostic factor for predicting recurrence and survival in melanoma patients.^{1,3} The exposure of the SLN to melanoma cells triggers an immune response (or lack thereof) that will be reflected in patterns of SLN gene expression. Although some individual specific genes were identified to be highly associated with prognosis of melanoma,^{8,9,14-16} there has been no systematic study that correlates prognosis to a panel of genes in the SLN that has the potential to be used in the clinic. The combinatorial assessment of multiple genes in the SLN can likely achieve increased sensitivity and specificity for risk evaluation.

Rationale for combining SLN signature with clinicopathologic factors as a prognostic model: Currently, melanoma prognosis is mostly based on traditional clinicopathological factors. There are no reliable biomarkers that can be implemented into current clinical prognostics for melanoma patients. It is believed that a prognostic gene expression signature of the SLN can be developed that reflects the host response to exposure to melanoma cells and predicts the likelihood of melanoma recurrence. While such a gene signature, by itself, is contemplated to have independent prognostic significance, another model that further includes well-characterized clinicopathologic prognostic factors (e.g., Breslow thickness, ulceration, number of positive nodes) is contemplated.

Optimization of SLN Biomarker and Validation Independent Patient Set.

Sample stratification: Stratified sampling on age and gender are used to maintain the population proportions to select RNA samples from tumor-positive SLN patients with 1:1 sampling between the outcome, recurrence, and no recurrence for a sample size of 80 (n₁=40, n₂=40) in the training set and 60 patients' samples in the validation set (n₁=30, n₂=30). A small divergence between the expected sampling distribution and actual distribution in cases is expected. The statistically significant covariates not controlled for in the sampling is handled post-randomization in the statistical analyses through ANOVA/ANCOVA/GLM.

Microarray and real-time RT-PCR experiments: The microarray and real time RT-PCR experiment procedures and quality controls are similar to those described in Example 1. 80 patient samples from the most radioactive SLN (40 controls without recurrence and 40 cases with recurrence) are selected to do microarray to get a list of genes that are associated with recurrence or no recurrence. Quantitative real-time RT-PCR is performed to confirm the SLN gene signature. The list is refined to about 20 or fewer prognosis-related genes. Another 60 tumor-positive SLN RNA samples are selected from the Louisville Sentinel Lymph Node Biorepository (30 controls with no recurrence, 30 cases with recurrence) as a validation set to perform RT-PCR to validate the SLN gene signature. The gene signature identified in this validation set is compared with those identified in the training set. The selection of the final prognosis-related genes is based primarily on the strength of their performance in the training set and in this validation set. A final list will include about 20 or fewer prognosis-related genes.

Sample size and power calculations: About 80 more samples in the training set and 60 samples in the validation set is studied. The total number of samples is 160 including the sample numbers in the preliminary data (80 cases and 80 controls) in the training set and 60 samples in the validation set. Top 20 genes with an observed significant level <10⁻³ have already been identified. Significance level is set to α=10⁻⁴. At least 2-fold changes can be detected with more than 80% power.²² At False Discovery Rate (FDR) level of 10%, the revised significance level (α) is 0.007, which is higher than what is set in the justification above. To validate the findings of the microarray experiments, selected genes (about 20) are studied using RT-PCR. Five randomly selected genes are included that are not identified in the microarray experiment. In these RT-PCR experiments, n₁=40, n₂=40 in the training set and n₁=30, n₂=30 in the validation set is studied. With sample size of n₁=40 and n₂=40, using a two-sided, two-sample t test, at α=0.0011 (=0.05/45), β=0.20 (power=80%), 0.90SD units change in two groups (about 2-fold change) can be identified, which is a large effect size.

Statistical analyses: The microarray data is pre-processed using standard algorithms to ensure the reliability of the data and remove outlier arrays using Robust Multi-array Averaging.¹⁸ To determine differential expression in the first dataset, an ANOVA/GLM model analysis is utilized controlling for the stratification factors, known significant clinical predictors and for any experimental conduct differences such as microarray batches and known technical variation. Due to the high dimension of the data and the number of identical tests being performed, the false-positive rate is inflated. The false-positives are controlled for using the method of Benjamini-Hochberg.²³ Once set of DEGs has been identified sequentially sized subsets of the genes are used to predict the classification sensitivity and specificity of the gene set, using support vector machines, hierarchical clustering, and principal component analysis. To assess the performance of each of the sequentially sized classifiers, k-fold cross validation (such as 10-fold cross validation) is performed on a bootstraps samples (such as size 100).²⁴ These methods will show the ability of the genes to segregate the prognostic groups.²⁵⁻²⁷ A minimum gene set is chosen that maximizes both the classification sensitivity and specificity. The focus is on identifying the gene signature set of 20 or fewer genes, which gives us the best ability to discriminate between good versus poor prognosis patients. This gene signature set is aggregated into 1 or 2 components representing a network of complicit genes, using principal components analysis, and is used to predict overall prognosis along with clinicopathologic features.^{28,29} The genes identified are correlated with the biologically functions to rationale the gene list. Analysis of these samples will allow for narrowing the gene signature to a smaller subset of genes (about 20 genes) that are amenable to analysis using a multi-marker RT-PCR platform.

The statistical analysis for the RT-PCR experiments is very similar to the microarray analysis, but with limited number of genes. It is expected that none of the randomly selected genes that were added in the list and were not significant will appear in the top 10 genes.

It is contemplated that a prognostic gene signature in SLN-positive patients are identified and validated, which would have clinical utility in making adjuvant therapy decisions and for risk stratification in clinical trials. The prototype for this is the use of molecular gene profiling to direct adjuvant therapy in breast cancer.²¹

Prognostic Model that Incorporates the Gene Signature in Addition to Standard Clinicopathologic Factors.

The gene signatures that have been identified and validated are incorporated with clinicopathologic prognostic factors (e.g., Breslow thickness, ulceration, number of positive lymph nodes, etc.) to create a prognostic scoring system to distinguish the truly high-risk patients from the low-risk patients who may be spared from toxic adjuvant therapy.

Prognostic models are created based on the well-known Cox regression model, which has been widely accepted as an excellent model to study multivariable melanoma prognosis and modeling.³⁰⁻³⁵ The gene expression signature is counted as one-two predictor(s). It is included in the multivariable Cox regression analysis, along with the following well-known clinicopathologic features: age, gender, primary lesion site, Breslow thickness, ulceration, tumor burden (micro vs. macrometastasis), and the number of positive nodes. In the Cox regression analysis, gene expressions are covariates with huge variability. Therefore, some transformations are explored or categorical groups identified to reduce impact of variability in gene expression data on multivariable model. Significant prognostic factors identified in the multivariable analyses are included in the predictive model for SLN-positive patients. This prognostic model can be used to generate projected 5-year disease-free and survival rates for an individual patient along with standard error and 95% confidence interval.^{36,37}

On the basis of this predictive model, a prognostic scoring system representing an individual SLN-positive patient's prognosis can be generated based on the 5-year disease-free and survival rates, predicted by the model for that patient.^{36,37} For example, a patient is assigned a score of 60 if this patient's predicted 5-year survival rate is 60%. The projected 5-year disease-free and overall survival rates are proposed as a prognostic score, because in SLN-positive patients, 5 years of follow-up is generally considered sufficient; most events will have occurred by that time. Thus, the proposed prognostic score could be considered a composite prognostic indicator of several dominant prognostic factors in melanoma, and it represents the probability of a patient's long-term risk of recurrence and mortality.

It is expected that a practical patient risk classification system can be generated based on this prognostic scoring system. For example, based on prognostic scores, three patient risk groups can be defined as follows: patients with a prognostic score >70 are assigned to low-risk group, 40-70 as intermediate-risk group, and <40 as high-risk group. This patient risk classification system is obviously more accurate and useful compared to the traditional AJCC staging system, because it contains additional information on other significant prognostic factors as well as the gene signatures that cannot be used within the current constraints of the overall AJCC staging system criteria. Bootstrapping or cross-validation is used to validate the model unbiased.³⁸

Sample size and power calculations: There is a sample size of 160 (80 controls and 80 cases) in the training set and 60 (30 controls and 30 cases) in the validation set. Using the predicting model, all subjects are classified into three groups (L=Low Risk, I=Intermediate Risk, H=High Risk). Sensitivity, specificity, ROC is estimated based on comparing L+I vs. H and L vs I+H before proceeding to comparing ordered categories.³⁹ Lower alpha is used instead of 5% due to many comparisons. Using one sample binomial test, a true sensitivity of 0.90 can be detected from a minimum detectable limit of 0.75 at alpha=0.025 and with power of 85%. The sample size was estimated with the inclusion of other variables of interest (primary lesion site, Breslow thickness, ulceration, tumor burden, and number of positive nodes) in the Cox regression model with expected R of 0.30. The sample size of 220 will serve as a data set for model development.

Statistical analyses: Sensitivity, specificity, concordance correlation coefficient, ROC and AUC are estimated; a good practical example is demonstrated in SUGI 31;⁴⁰ another source is by Pepe.⁴¹ The prognostic model is validated using an independent validation data set. Concordance correlation coefficients are calculated on the basis of the direct comparisons of the predicted 5-year survival rate and the observed 5-year survival rate estimated from the validation data set. An Integrated Brier Score is calculated to assess the validity of the predictive model developed. This is further explored by combining the training set and validation set data together. Using the bootstrap approach, the data is split into two equal sets and evaluate predictive power.

The present inventors contemplate the prognostic scoring system for stratifying risk assessment in the SLN-positive melanoma patients by incorporating both SLN gene signatures and clinicopathological features. This model is unique in this field. Consultation in areas of in multivariate modeling of melanoma prognosis, statistics and bioinformatics can be conducted.

The overall impact of this work will: (1) promote the best quality of life of melanoma patients by avoiding unnecessary toxic treatment in a “low-risk” group of patients and increase the overall survival rate by identifying a truly “high-risk” group of patients who are most likely to benefit from adjuvant therapy; (2) allow more precise stratification of patients for entry of homogeneous risk groups into clinical trials of novel adjuvant therapies in the era of SLN biopsy—a problem that has plagued some adjuvant therapy trials in the past.

Throughout this document, various references are mentioned. All such references are incorporated herein by reference to the same extent as if each individual reference was specifically and individually indicated to be incorporated by ref, including the references set forth in the following list:

### REFERENCES

1. de Vries M, Speijers M J, Bastiaannet E, Plukker J T, Brouwers A H, van Ginkel R J, Suurmeijer A J, Hoekstra H J. Long-term follow-up reveals that ulceration and sentinel lymph node status are the strongest predictors for survival in patients with primary cutaneous melanoma. Eur J Surg Oncol. 2011, 37(8):681-7. Epub 2011 Jun. 1. PMID: 21636244

2. de Giorgi V, Grazzini M, Massi D. Sentinel-lymph-node biopsy for cutaneous melanoma. N Engl J Med. 2011, 365(6):570-71 PMID: 21830976

3. Balch C M, Gershenwald J E, Soong S J, Thompson J F. Update on the melanoma staging system: The importance of sentinel node staging and primary tumor mitotic rate. J Surg Oncol. 2011, 104(4):379-85. doi: 10.1002/jso.21876. PMID: 21858832

4. Averbook B J. Mitotic rate and sentinel lymph node tumor burden topography: integration into melanoma staging and stratification use in clinical trials. J Clin Oncol. 2011, 29(16):2137-41. Epub 2011 Apr. 25. PMID: 21519024

5. Berger A C, Fierro M, Kairys J C, Berd D, Sato T, Andrei J, Hyslop T, Mastrangelo M J.

Lymph node ratio is an important and independent prognostic factor for patients with stage III melanoma. J Surg Oncol. 2011 Aug 3. doi: 10.1002/jso.22051. PMID: 21815149

6. Balch C M, Gershenwald J E, Soong S J, Thompson J F, Ding S, Byrd D R, Cascinelli N, Cochran A J, Coit D G, Eggermont A M, Johnson T, Kirkwood J M, Leong S P, McMasters K M, Mihm M C Jr, Morton D L, Ross M I, Sondak V K. Multivariate analysis of prognostic factors among 2,313 patients with stage III melanoma: comparison of nodal micrometastases versus macrometastases. J Clin Oncol. 2010, 28(14):2452-9. PMID: 20368546

7. McMasters et al, Final results of the Sunbelt Melanoma Trial, ASCO annual meeting 2008; Abstract #9003.

8. Winnepenninckx V, van den Oord J J. Gene expression profiling and clinical outcome in melanoma: in search of novel prognostic factors. Expert Rev Anticancer Ther. 2007, 7(11):1611-31. PMID: 18020928

9. Tímír J, Gyorffy B, Rásó E. Gene signature of the metastatic potential of cutaneous melanoma: too much for too little? Clin Exp Metastasis. 2010 Aug; 27(6):371-87. PMID: 20177751

10. Segura M F, Belitskaya-Lévy I, Rose A E, Zakrzewski J, Gaziel A, Hanniford D, Darvishian F, Berman R S, Shapiro R L, Pavlick A C, Osman I, Hernando E. Melanoma MicroRNA signature predicts post-recurrence survival. Clin Cancer Res. 2010,16(5):1577-86. PMID: 20179230

11. Hardesty W M, Kelley M C, Mi D, Low R L, Caprioli R M. Protein signatures for survival and recurrence in metastatic melanoma. J Proteomics. 2011, 74(7):1002-14. PMID: 21549228

12. Reintgen D, Cruse C W, Wells K, et al: The orderly progression of melanoma nodal metastases. Ann Surg 1994, 220:759-767

13. Krag D N, Meijer S J, Weaver D L, et al: Minimal-access surgery for staging of malignant melanoma. Arch Surg 1995, 130:654-658

14. Melanoma prognosis: a REMARK-based systematic review and bioinformatic analysis of immunohistochemical and gene microarray studies. Schramm S J, Mann G J. Mol Cancer Ther. 2011, 10(8):1520-8. Epub 2011 Jun. 9.PMID:21659462

15. Bogunovic D, O'Neill D W, Belitskaya-Levy I, Vacic V, Yu Y L, Adams S, Darvishian F, Berman R, Shapiro R, Pavlick A C, Lonardi S, Zavadil J, Osman I, Bhardwaj N. Immune profile and mitotic index of metastatic melanoma lesions enhance clinical staging in predicting patient survival. Proc Natl Acad Sci USA. 2009, 106(48):20429-34. Epub 2009 Nov. 13. PMID:19915147

16. Mandruzzato S, Callegaro A, Turcatel G, Francescato S, Montesco M C, Chiarion-Sileni V, Mocellin S, Rossi C R, Bicciato S, Wang E, Marincola F M, Zanovello P. A gene expression signature associated with survival in metastatic melanoma. J Transl Med. 2006, 4:50. PMID: 17129373

17. McMasters K M, Noyes R D, Reintgen D S, Goydos J S, Beitsch P D, Davidson B S, Sussman J J, Gershenwald J E, Ross M I, Sunbelt Melanoma Trial. Lessons learned from the Sunbelt Melanoma Trial. J Surg Oncol 2004; 86(4): 212-233

18. Bolstad B M, Irizarry R A, Astrand M, and Speed T P. A comparison of normalization methods for high density oligonucleotide array data based on variance and bias. Bioinformatics 2003; 19: 185-93

19. Tusher V G, Tibshirani R, and Chu G. Significance analysis of microarrays applied to the ionizing radiation response. Proc Natl Acad Sci USA 2001, 98: 5116-21

20. ArrayVigil: a methodology for statistical comparison of gene signatures using segregated-one-tailed (SOT) Wilcoxon's signed-rank test. Khan H A. J Mol Biol. 2005, 345(4):645-9.

PMID: 15588814

21. Goldstein U, Gray R, Badve S, Barrett H C, Yoshizawa C, Rowley S, et al. Prognostic utility of the 21-gene assay in hormone receptor-positive operable breast cancer compared with classical clinicopathologic features. J Clin Oncol 2008, 26(25): 4063-71

22. Jung Sin-Ho. Sample size for FDR-control in microarray data analysis. Bioinformatics, 2005, 21:3097-3104

23. Benjamini Y, Drai D, Elmer G., Kafkafi N, and Golani I. Controlling the false discovery rate in behavior genetics research. Behav Brain Res 2001, 125: 279-84

24. Hastie T., The elements of statistical learning; data mining, inference and prediction 2001, New York, N.Y.: Springer. 214-221.

25. Ancona N., et al., On the statistical assessment of classifiers using DNA microarray data. BMC Bioinformatics, 2006. 7 (387).

26. Rojas, J., et al., Development of Predictive Models of Proliferative Vitreoretinopathy Based on Genetic Variables. The Retina 4 Project. Invest Ophthalmol Vis Sci, 2008.

27. Zhang, H., et al., Support vector machine and pharmacophore-based prediction models of multidrugresistance protein 2 (MRP2) inhibitors. Eur J Pharm Sci, 2008.

28. Hargrove U, Li G, Englehart K B, Hudgins B S. Principal components analysis preprocessing for improved classification accuracies in pattern-recognition-based myoelectric control. IEEE Trans Biomed Eng. 2009 May; 56(5):1407-14.

29. Song J J, Ren Y, Yan F. Classification for high-throughput data with an optimal subset of principal components. Comput Biol Chem. 2009, 33(5):408-13.

30. Haqq C, Nosrati M, Sudilovsky D, et al. The gene expression signatures of melanoma progression. Proc Natl Acad Sci USA 2005; 102: 6092-7.

31. Sondak V K, Messina J L. Current status of biomarkers for melanoma metastasis. IDrugs. 2006, 9(9): 627-31

32. Torisu-Itakura H, Lee J H, Scheri R P, Huynh Y, Ye X, Essner R, Morton D L. Molecular characterization of inflammatory genes in sentinel and nonsentinel nodes in melanoma. Clin Cancer Res. 2007, 13(11):3125-32

33. Kounalakis N, Goydos J S. Tumor cell and circulating markers in melanoma: diagnosis, prognosis, and management. Curr Oncol Rep. 2005; 7(5): 377-82

34. Ludwig J A et al, Biomarkers in cancer staging, prognosis and treatment selection. Nat Rev Cancer 2005, 5:845-56

35. Torabian S et al, Biomarkers for melanoma. Curr Opin Oncol 2005, 17:167-71

36. Soong S J, Ding S, Coit D, Balch C M, Gershenwald J E, Thompson J F, Gimotty P; AJCC Melanoma Task Force. Predicting survival outcome of localized melanoma: an electronic prediction tool based on the AJCC Melanoma Database. Ann Surg Oncol. 2010, 17(8):2006-14. PMID: 20379784

37. Soong S J, Ding S, Coit D, Balch C M. Chapter 5: Models for predicting melanoma outcome. In Cutaneous Melanoma (5th Edition). Balch C M, Houghton A N, Sober A J, Soong S J, Atkins M B, Thompson J F, eds. 2009, Quality Medical Publishing, Inc., St. Louis, Mo.; pp 87-104.

38. Frank E, Harrell Jr., Kerry Lee, Daniel Mark. Multivariable prognostic models: issues in developing models, evaluating assumptions and adequacy, and measuring and reducing errors. Statistics in Medicine, 1996, 15(4): 361-387

39. Agresti A. Categorical data analysis, 2nd ed. New York: Wiley and Sons, 2002; Agresti 2002, Fleiss J L, Levin, B and Paik M C. Statistical Methods for Rates and Proportions, 3rd Edition. New York: John Wiley and Sons, 2003

40. Mithat Gönen Receiver Operating Characteristic (ROC) Curves. SUGI 31 Proceedings Page 210-31

41. Pepe M S. The Statistical Evaluation of Medical Tests for Classification and Prediction. Oxford Statistical Science Series, 2003

42. McMasters K M, Reintgen D S, Ross M I, et al: Sentinel lymph node biopsy for melanoma: How many radioactive nodes should be removed? Ann Surg Oncol 8:192-197, 2001

43. McMasters K M. The Sunbelt Melanoma Trial. Ann Surg Oncol 2001; 8:41s-43s.

44. Reynolds S R, Albrecht J, Shapiro R L, et al: Changes in the presence of multiple markers of circulating melanoma cells correlate with clinical outcome in patients with melanoma. Clin Cancer Res 9:1497-1502, 2003.

45. Y. Benjamini and Y. Hochberg (1995). Controlling the false discovery rate: a practical and powerful approach to multiple testing. J. R. Statist. Soc. B. Vol. 57: 289-300.

It will be understood that various details of the presently disclosed subject matter can be changed without departing from the scope of the subject matter disclosed herein. Furthermore, the foregoing description is for the purpose of illustration only, and not for the purpose of limitation.

1.-107. (canceled)

108. A method for detecting RNA in a human subject with melanoma comprising:
obtaining a sample from a sentinel lymph node (SLN) of the human subject;
detecting RNAs of at least one biomarker contained in the sample by contacting the sample with a biomarker-specific polynucleotide probe, where the at least one biomarkers comprises (a) polymeric immunoglobulin receptor (PIGR), transcription factor AP-2 alpha (activating enhancer binding protein 2 alpha) (TFAP2A), or both and (b) optionally one or more of ATP-binding cassette, sub-family B MDR/TAP, member 5 (ABCB5), mucin 7, secreted (MUC7), melan-A (MLANA), v-erb-b2 erythroblastic leukemia viral oncogene homolog 3 (ERBB3), paired box 3 (PAX3), dopachrome tautomerase (DCT), CD69 molecule (CD69), MOP-1, CD163 molecule (CD163), interferon-induced protein with tetratricopeptide repeats 1 (IFIT1), dual specificity phosphatase 1 (DUSP1), thrombospondin 1 (THBS1), interleukin 1, beta (IL1B), superoxide dismutase 2, mitochondrial (SOD2), prostaglandin-endoperoxide synthase 2 (prostaglandin G/H synthase and cyclooxygenase) (PTGS2), regulator of G-protein signaling 2, 24 kDa (RGS2), regulator of G-protein signaling 1 (RGS1), or nuclear receptor subfamily 4, group A, member 2 (NR4A2); and
detecting the binding between the RNAs and the polynucleotide probes, wherein the detecting is not carried out using a microarray.

109. The method of claim 108, wherein the at least one biomarkers comprises PIGR and TFAP2A.

110. The method of claim 108, wherein the biomarker-specific polynucleotide probe consists of a biomarker-specific polynucleotide probe for each of the biomarkers ABCB5, PIGR, TFAP2A, MUC7, MLANA, ERBB3, PAX3, DCT, CD69, MOP-1, CD163, IFIT1, DUSP1, THBS1, IL1B, SOD2, PTGS2, RGS2, RGS1, and NR4A2.

111. The method of claim 108, wherein the at least one biomarkers consist of ABCB5, TFAP2A, MUC7, PIGR, ERBB3, PAX3, RGS2, and IL1B.

112. The method of claim 108, wherein the at least one biomarkers consist of (a) PIGR, TFAP2A, or both and (b) optionally one or more of ABCB5, MUC7, MLANA, ERBB3, PAX3, DCT, CD69, MOP-1, CD163, IFIT1, DUSP1, THBS1, IL1B, SOD2, PTGS2, RGS2, RGS1, or NR4A2.

113. The method of claim 108, wherein the at least one biomarkers consist of:
(a) PIGR, TFAP2A, or both and (b) optionally one or more of ABCB5, MUC7, PAX3, CD69, MOP-1, CD163, IFIT1, DUSP1, THBS1, IL1B, SOD2, PTGS2, RGS2, RGS1, or NR4A2;
RGS1, RGS2, PIGR, CD69, ERBB3, and SOD2;
TFAP2A and PIGR;
RGS2, TFAP2A, PIGR, CD69, SOD2, ABCB5, NR4A2, and MUC7; or
RGS2, PIGR, MUC7, ABCB5, NR4A2.

114. The method of claim 108, wherein the at least one biomarkers consist of TFAP2A and PIGR.

115. The method of claim 108, wherein the human subject with melanoma has been diagnosed with stage III melanoma.

116. The method of claim 108, wherein the human subject is being treated with adjuvant therapy.

117. The method of claim 108, wherein the human subject is being treated with adjuvant therapy for treating melanoma.

118. The method of claim 108, wherein the human subject is being treated with interferon-alfa-2b (IFN) therapy.

119. The method of claim 108, wherein the method further comprises assessing a clinicopathologic feature of the human subject from which the sample was obtained.

120. The method of claim 118, wherein the clinicopathologic feature is selected from: age, gender, anatomic location, Breslow thickness, ulceration, and sentinel lymph node status.

121. The method of claim 118, wherein the clinicopathologic feature is selected from: metastasis, age, lesion site, tumor burden, number of positive nodes, ulceration, and tumor thickness.

122. The method of claim 108, wherein the human subject has a high risk of recurrence of melanoma when there is an increased binding of the RNAs and polynucleotide probes relative to a control sample obtained from an SLN of a human subject with melanoma who had no recurrence for at least 5 years.

123. The method of claim 108, wherein the method further comprises treating the human subject with adjuvant therapy.

124. The method of claim 108, wherein the method further comprises treating the human subject with adjuvant therapy for treating melanoma.

125. The method of claim 108, wherein the method further comprises treating the human subject is treated with interferon-alfa-2b (IFN) therapy.

126. The method of claim 108, wherein the detecting is carried out by one or a combination of polymerase chain reaction, real-time polymerase chain reaction, reverse transcriptase polymerase chain reaction, or real-time quantitative RT-PCR.

127. A method for treating melanoma in a human patient, the method comprising:
(1) obtaining a sample from a sentinel lymph node of the human patient;
(2) quantifying an RNA expression level for at least one biomarkers contained in the sample, the at least one biomarkers consisting of polymeric immunoglobulin receptor (PIGR) and transcription factor AP-2 alpha (activating enhancer binding protein 2 alpha) (TFAP2A) wherein the quantifying is carried out by one or a combination of polymerase chain reaction, real-time polymerase chain reaction, reverse transcriptase polymerase chain reaction, real-time quantitative RT-PCR, or probe array; and
(3) treating the human patient with adjuvant therapy.

128. The method of claim 127, wherein the human subject is treated with adjuvant therapy for treating melanoma.

129. The method of claim 127, wherein the human subject is treated with interferon-alfa-2b (IFN) therapy.

130. The method of claim 127, wherein the quantifying is carried out by one or a combination of polymerase chain reaction, real-time polymerase chain reaction, reverse transcriptase polymerase chain reaction, or real-time quantitative RT-PCR.

131. The method of claim 127, wherein the quantifying is not carried out by a microarray.

132. The method of claim 127, wherein the quantifying is relative to a sample from a human subject having melanoma without recurrence for at least 5 years.

133. A method for treating melanoma in a human patient, the method comprising:
(1) obtaining a sample from a sentinel lymph node of the human patient;
(2) quantifying an RNA expression level for at least one biomarkers contained in the sample, where the at least one biomarkers consists of (a) polymeric immunoglobulin receptor (PIGR) and transcription factor AP-2 alpha (activating enhancer binding protein 2 alpha) (TFAP2A) and (b) optionally one or more of ATP-binding cassette, sub-family B MDR/TAP, member 5 (ABCB5), mucin 7, secreted (MUC7), melan-A (MLANA), v-erb-b2 erythroblastic leukemia viral oncogene homolog 3 (ERBB3), paired box 3 (PAX3), dopachrome tautomerase (DCT), CD69 molecule (CD69), MOP-1, CD163 molecule (CD163), interferon-induced protein with tetratricopeptide repeats 1 (IFIT1), dual specificity phosphatase 1 (DUSP1), thrombospondin 1 (THBS1), interleukin 1, beta (IL1B), superoxide dismutase 2, mitochondrial (SOD2), prostaglandin-endoperoxide synthase 2 (prostaglandin G/H synthase and cyclooxygenase) (PTGS2), regulator of G-protein signaling 2, 24 kDa (RGS2), regulator of G-protein signaling 1 (RGS1), or nuclear receptor subfamily 4, group A, member 2 (NR4A2), wherein the quantifying is carried out by one or a combination of polymerase chain reaction, real-time polymerase chain reaction, reverse transcriptase polymerase chain reaction, real-time quantitative RT-PCR, or probe array; and
(3) treating the human patient with interferon-alfa-2b (IFN) therapy.
